(19) United States
(12) Patent Application Publication
(10) Pub. No.: US 2005/0043640 A1
(43) Pub. Date: Feb. 24, 2005

(54) Remote electrocardiogram for early detection of coronary heart disease

(76) Inventor: Alexander C. Chang, Palm Springs, CA (US)

Correspondence Address: MORGAN, LEWIS & BOCKIUS, LLP., 2 PALO ALTO SQUARE, 3000 EL CAMINO REAL, PALO ALTO, CA, 94306, US

(21) Appl. No.: 10/647,161

(22) Filed: Aug. 21, 2003

(51) Int. Cl. A61B 5/0402

(52) U.S. Cl. 600/509

(57) ABSTRACT

An apparatus for electrocardiogram measurement including a first non-conductive pad. A first and second electrode are disposed on the first non-conductive pad. The first electrode represents any one of V₄, V₅, or V₆ and the second electrode is either (i) positioned on the subject below the first electrode in order to represent left leg (LL) or (ii) is placed on a line on the subject defined by the V₄, V₅, and V₆ precordial positions in order to represent any one of V₄, V₅, or V₆ not represented by the first electrode. The apparatus includes a second non-conductive pad. A third electrode is disposed on the second non-conductive pad. The third electrode is a right arm (RA) electrode that is for positioning at a position that is on or close to the right arm of the subject. Each electrode is adapted for electrical connection with the skin in order to receive and transmit electrical impulses. The apparatus further includes an electrical connection that connects each electrode disposed on the first and second non-conductive pads to an electrocardiological measuring apparatus. The electrocardiological measuring apparatus is capable of measuring a first lead and a different second lead without user intervention.

### 1. FIELD OF THE INVENTION

This invention relates to an apparatus used to monitor and record the electrical activity produced by the human heart. This invention further relates to systems and methods for analyzing electrical activity produced by the human heart in a remote system for pre-screening identification purposes. Such systems and methods can be used as part of a comprehensive program designed to improve health care and to lower costs associated with human conditions such as coronary heart disease.

### 2. BACKGROUND OF THE INVENTION

Despite improved clinical care, heightened public awareness, and widespread use of health innovations, coronary heart disease (CHD) remains the leading cause of death in the United States (American Heart Association, Heart Disease and Stroke Statistics—2003 Update, www.americanheart.org), and the decline in rates from CHD that began during the 1960s slowed during the 1990s. CHD not only devastates individuals and families, it costs the United States economy billions a year in medical expenses and lost productivity. Because of these vast utilization costs, early detection of disease risk that, in turn, enables early intervention with therapeutics and lifestyle changes is needed, as it is a proven method to reduce patient morbidity and mortality rate. See, for example, Guide to Clinical Preventive Services, second edition, 1996, Report of the U.S. Preventive Services Task Force, U.S. Department of Health and Human Services. Accordingly, a comprehensive screening strategy that screens for modifiable cardiac risk factors, such as hypertension, elevated serum cholesterol, cigarette smoking, obesity, physical activity, diet, etc., coupled with detection of silent and inducible ischemia asymptomatic CHD(CAD) employing a sensitive ECG test is needed in the art.

While clinicians emphasize proven measures for the primary prevention of coronary disease, current strategies to refer patients with asymptomatic CHD to clinicians are inadequate because the patients exhibit no outward signs of symptoms and therefore do not go to the appropriate medical clinicians for evaluation. Thus, a pre-screening strategy that can capture coronary risk factors and ECG readings for a large population that is both efficient and exhibits minimum clinical overhead, that extends access to essential clinical services, and provides a means to encourage participation in taking ownership of modifiable risk factors is needed in the art.

### 2.1 Electrocardiograms

An electrocardiogram involves the use of body surface electrodes that are non-invasively coupled to a patient's intrinsic cardiac electrical activity using various types of medical, diagnostic, and therapeutic equipment. Electrical signals generated by the human heart appear in a characteristic pattern throughout the body and on its surface. Such intrinsic cardiac electrical activity can be measured by placing electrodes on the skin of the individual and measuring the voltage between a particular electrode and a reference potential or between selected bipolar pairs of electrodes. The technique of using skin electrodes to sense changes in electrical potential caused by polarization and depolarization of the heart as it beats has been in use for over one hundred years.

Well known bipolar pairs are typically located on a patient's right arm (RA), left arm (LA), right leg (RL) (commonly used as a reference), and left leg (LL). Monopolar electrodes referenced properly are referred to as V leads and are positioned anatomically on a patient's chest according to an established convention. In heart monitoring and diagnosis, the voltage differential appearing between two electrodes or between one electrode and the average of a group of other electrodes represents a particular perspective of the heart's electrical activity and it is this differential, or collection of differentials, that are generally referred to as an electrocardiogram (ECG or EKG).

### 2.2 Electrocardiogram Leads

Particular combinations of electrodes are called leads. For example, the three bipolar limb leads are:
Lead I=(LA−RA)
Lead II=(LL−RA)
Lead III=(LL−LA)

Leads I, II, and III are illustrated in FIG. 1. In addition to leads I, II, and III, five-electrode systems are used to capture signals. Such five-electrode systems include one precordial and four limb electrodes (LA, RA, LL, and RL). The precordial electrodes are placed at six positions across the precordium as illustrated in FIG. 2A.
Lead V₁=V₁−(LA+RA+RL)/3
Lead V₂=V₂−(LA+RA+RL)/3
Lead V₃=V₃−(LA+RA+RL)/3
Lead V₄=V₄−(LA+RA+RL)/3
Lead V₅=V₅−(LA+RA+RL)/3
Lead V₆=V₆−(LA+RA+RL)/3

Additional leads that are used include:
Lead AVL=(LA+RA)/2−LL
Lead AVF=(LA+LL)/2−RA
Lead AVR=(RA+LL)/2−LA

In one application, electrical signals produced by the heart are transferred by electrodes to a monitoring apparatus known as an electrocardiograph for further processing. Unlike the four limb electrodes, placement of the six precordial electrodes V₁, V₂, V₃, V₄, V₅, and V₆ must be exact in order to insure the acquisition of signals that will have universal diagnostic meaning. If the V electrodes are not positioned properly or if they do not make good contact with the patient's skin, the recorded data may be invalid. Specifically, as illustrated in FIG. 2A, the V₁-V₆ electrodes are placed as follows:
V₁—in the fourth intercostal space at the right sternal border;
V₂—in the fourth intercostal space at the left sternal border;
V₄—in the fourth intercostal space at the mid-clavicular line;
V₃—midway between the V₂ and V₄ electrodes;
V₅—in the fifth intercostal space at the anterior auxiliary line;
V₆—in the fifth intercostal space in the mid-auxiliary line.

Any ECG that uses an unconventional system of leads necessarily detracts from the body of the experience that has been developed, in the interpretations of conventional ECGs, and can therefore be considered generally undesirable. The tracings generated would be understandable only by a relative few who were familiar with the unconventional system. Nevertheless, other lead systems have evolved from improvements in instrumentation that have permitted extension of electrocardiography to ambulatory, and even vigorously exercising subjects—and to recordings made over hours, or even days. For example, the limb electrodes can be moved from the arms to the trunk. Electrode placement using modified positions also requires medical training.

### 2.3 The Twelve-Lead Electrocardiogram

The “twelve-lead” ECG represents the gold-standard in the art of electrocardiograms. It has maximum sensitivity, but unfortunately, requires extensive medical training to properly implements and therefore is not a suitable screening tool for a large outpatient population. Nevertheless, the “twelve-lead” ECG has long been an important diagnostic tool in the field of cardiology. A “twelve-lead” ECG requires the individual placement of ten individual electrodes on the patient's body, six precordially (V₁-V₆) and one on each of the four limbs. The ten electrodes are attached one at a time and must each be placed over a specific point on the patient's body. If any of the precordial electrodes are mixed up, or if the arm or leg electrodes are swapped, the ECG tracing obtained will be faulty.

### 2.4 Electrocardiogram Sensitivity

Features of the ECG waveform, appearing as deflections from the resting electrical potential or baseline, are named. As illustrated in FIG. 2B, a heartbeat begins with the P wave, a small deflection indicating the beginning of the depolarization (contraction) sequence originating in the atrium. The connected series of peaked deflections labeled QRS is composed of the Q, the R and the S waves and is refereed to as the QRS complex. The QRS complex corresponds to the depolarization of the ventricles. It is followed by the T wave. The T wave amplitude is normally less than that of the QRS complex and wider. The T wave corresponds to the repolarization of the heart in preparation for the next beat. It is normally followed by a short period of resting potential.

In order to detect CHD, it is necessary to detect ischemic ST-segment changes. The ST-segment is a particular part of the electrocardiographic waveform, which is illustrated in FIG. 2B. See, for example, Dahser and Slye, ECG, arrhythmia, and ST segment analysis, Redmond, Wash., 1994, SpaceLabs Medical, Inc.). Each lead has a different sensitivity at detecting such ischemic ST-segment changes. To investigate lead sensitivity, London et al. evaluated a large cohert of patients undergoing major noncardiac surgery using a 12-lead ECG. Fifty-one ischemic episodes were detected among 105 patients. The sensitivity of each of the leads at detecting these 51 episodes, where sensitivity is defined as the percentage of the 51 episodes detected, is illustrated in FIG. 2C.

As illustrated in FIG. 2C, the V₅ lead has a 75% sensitivity relative to the 12-lead ECG and the V₄ lead has a 61% sensitivity relative to the 12-lead ECG. In contrast, leads V₃ and V₆ are significantly less sensitive, with sensitivities of approximately 23% and 38%, respectively, relative to the 12-lead ECG. See London et al., 1988, Anesthesiology 69, p. 232-241. Furthermore, Table 1 illustrates the sensitivity for different ECG lead combinations based on the data presented in London et al.

TABLE 1
Sensitivity for different ECG lead combinations

| Lead Combination | | Sensitivity (%) |
|---|---|---|
| 1 lead | II | 33 |
| | V₄ | 61 |
| | V₅ | 75 |
| 2 leads | II/V₅ | 80 |
| | II/V₄ | 82 |
| | V₄/V₅ | 90 |
| 3 leads | V₃/V₄/V₅ | 94 |
| | II/V₄/V₅ | 96 |
| 4 leads | II/V₂-V₅ | 100 |

The variance in lead sensitivity imposes an additional constraint on electrocardiogram apparatus and techniques that are suitable for screening a large population. In addition to minimizing the amount of medical or technical training necessary to make the ECG measurements, the ECG apparatus must support leads that have maximum sensitivity.

### 2.5 Alternative Electrocardiogram Designs

To attempt to reduce the cost of ECG measurement and to broaden the practical application of ECG reduced-lead ECG systems have been designed. U.S. Pat. No. 4,583,549 to Manoli describes an ECG electrode pad in which the six precordial electrodes (V₁ through V₆) are plated and etched on an adhesive pad in a pattern designed to place these electrodes at the correct precordial positions (FIG. 3A). In FIG. 3A, electrodes 5, 15, 25, 35, 45, and 55 are respectively the V₁, V₂, V₃, V₄, V₅, and V₆ electrodes and they are all positioned on a common pad 1. Manoli contemplates that, considering the range of sizes of individuals, a great percentage of all patients can be tested by the use of three different pad sizes, namely pediatric, medium size adult and large size adult. The drawback with Manoli is that such systems still require extensive medically training in order to obtain useful data. As such, Manoli is not well suited for the screening of a community for signs of CHD.

U.S. Pat. No. 4,121,575 to Mills et al. describes a similar multiple electrode device, formed in stretchable non-conductive material having apertures in the V₁-V₆ positions. However, like the systems described in Manoli, the systems described in Mills et al include numerous electrodes and therefore require trained medical personal to properly address such electrodes.

U.S. Pat. Nos. 4,328,814 and 5,341,806 disclose ECG strips in which individual electrodes are physically connected to one another through bundled conductors terminating in a connector block. The drawback with such ECG strips is that a medical technician is needed because the bundling of conductors as described in these patents does not materially improve positioning of the electrodes, as each must be individually placed on the chest of a subject. As such, these devices are not suitable for screening a population signs of CHD because too much medical assistance is needed to use such devices.

U.S. Pat. No. 4,957,109 to Groeger et al. describes an electrode assembly comprising right and left arm and leg leads, and precordial leads all affixed to a common structure. The arm and leg leads do not affix to a patient's chest during use. However, like the Mills et al. system, the Groeger et al. system does not serve to maintain a relatively fixed positioning of electrodes therein during use.

U.S. Pat. No. 5,184,620 to Cudahy et al. describes an electrode pad system comprised of a multiplicity of electrodes that are utilized in defibrillation and pacing scenarios as directed by an on-line computer driven analysis and electrical energy application system. This system distributes electrical energy to appropriate sets of the multiplicity electrodes in response to patient needs.

Other related art is disclosed in U.S. Pat. Nos. 5,507,290; 5,327,888; 5,042,481; 4,852,572; and 4,763,660. The devices disclosed in these patents suffer one or more limitations such as lack of precise repositioning ability, failure to intimately follow chest curvatures and/or cross talk between ECG leads. These devices are not at all applicable for self-ECG testing.

### 2.6 Three Lead Electrocardiogram Designs

One form of ECG system that is relatively easy to use is the bipolar three lead monitor system illustrated in FIG. 3B. The system includes an electrode 302 for the right arm (RA), an electrode 304 for the left arm (LA), and an electrode 306 for the left leg (LL). The bipolar three lead monitor system is capable of measuring leads I, II, and III. However, such leads do not have sufficient sensitivity to detect ischemia. For example, lead II has an ischemia detection sensitivity of 33%. See London et al., 1988, Anesthesiology 69, p. 232-241. Thus, although bipolar three lead monitor systems do not require substantial medical personal to use, they are not suited for screening populations for symptoms of CHD, such as ischemia.

### 2.7 Five Lead Electrocardiogram Designs

Commercially available five lead monitoring systems provide improved sensitivity relative to bipolar three lead monitor systems because they allow for the monitoring of one precordial lead (any of V₁ to V₆). The electrode configuration for such commercially available five lead monitoring systems is illustrated in FIG. 3C. In addition to an electrode 302 (RA), an electrode 304 (LA), and an electrode 306 (LL) that are present in the bipolar three lead monitor system, the five lead monitoring system includes an electrode 310 for the right leg, and an electrode 314 that is positioned at any one of the precordial positions (any of V₁ to V₆).

The drawback with five lead monitoring systems is that, in order to support one precordial lead, five electrodes are required. This is a substantial drawback because the correct positioning of five electrodes is inconvenient. Furthermore, such five lead monitoring systems only provides a single lead (e.g., a precordial lead). Thus, another drawback with five lead monitoring systems as illustrated in FIG. 3C is that they do not provide satisfactory detection of ischemia.

### 2.8 Ambulatory Electrocardiogram Designs

FIG. 3D illustrates a commercially available ambulatory ECG system that has seven leads. In addition to an electrode 302 (RA), an electrode 304 (LA), an electrode 306 (LL), and an electrode 310 (RL) that are present in the five lead monitoring systems illustrated in FIG. 3C, the ambulatory ECG system has two precordial electrodes (electrode 314 and 318) and at electrode 320 that is placed on a patient at a position known as the M position.

The ambulatory ECG system illustrated in FIG. 7 allows for the monitoring of leads such as Lead II, CM5, and any two of V₁ through V₆. The device can also measure leads such as Lead I, Lead III, aVR, aVL, and aVF. However, a drawback with the ambulatory ECG system is that, with seven electrodes, it is not very easy to use and is, therefore, not a suitable system for screening a population for stress signs such as ischemia.

### 2.9 Modified V₅ (CS₅) Lead Systems

FIG. 3E illustrates a modified V₅ system that is commonly used by anesthesiologists during surgery or by cardiologists for ambulatory ECG monitoring. In the modified V₅ system, the left arm lead 364 is placed over the precordial V₅ position. The right arm lead 360 is placed on the right arm area (on the right arm or in the vicinity of the right arm). Finally, the modified V₅ system includes a left leg electrode that is placed below electrode 364 as illustrated in FIG. 3E. This lead is obtained by selecting lead I on a standard bipolar three-lead monitor. The configuration illustrated in FIG. 3E allows for the measurement of Lead II as well as the V₅ Lead. Lead II and the V₅ lead together have 75% of the ischemia detection sensitivity of a 12-lead ECG. However, the drawback with the modified V₅ system is that it is necessary to manually switch between Lead II and the V₅ lead and select Lead I on a monitor in order to get such signals. This is inconvenient. In addition, the modified V₅ system still requires the placement of three electrodes on three different pads on a subject. For these reasons, the modified V₅ configuration illustrated in FIG. 3E is not suited for use in general ECG screening regimens. The V₅ lead by itself does not have sufficient sensitivity, switching between Lead II and V₅ requires manual intervention, and there are three electrodes that must each be independently positioned in a correct manner.

### 2.10 State of the Art

Given the above background, it is apparent that devices in the known art do not provide devices that can be used for pre-screening that have sufficient sensitivity for detecting ischemia. Devices such as the three lead electrocardiogram are easy to use but do not have sufficient sensitivity. Devices such as the 12-lead ECG have the requisite sensitivity, but cannot be used for pre-screening. Thus, what is needed in the art are improved systems and methods for pre-screening for a CHD at-risk population.

### 3. SUMMARY OF THE INVENTION

The present invention provides novel pre-screening strategies that expand the number of capture points beyond that of the cardiologist or primary care physician offices to many diverse clinician offices. In this way, Internet-enabled data collection and ECG readings are not tied to a specific coronary heart disease symptom generated office visit. Rather, in accordance with the present invention, screening data is collected as a convenient procedure during any office visit, pre-employment physical, or even at other health care points where a minimum level of medical assistance is available. This strategy lowers cost of administration, while capturing the greatest number of patients for pre-screening management in an electronic database for ongoing disease management and clinician intervention follow-up.

Central to the pre-screening strategies of the present invention are novel ECG devices that feature ease of use, high sensitivity, digital portability of measured data, and widespread availability. The pre-screening strategies are further enabled by reaching out to screened individuals to encourage them to seek advice and increase knowledge of at-risk conditions via Internet website interaction, counseling, education, and primary healthcare provider communication. Furthermore, identification of those at high risk of asymptomatic coronary heart disease helps guide treatment decisions by care providers. (e.g., use of aspirin, cholesterol lowering drugs, blood pressure lowering drugs, control diabetes, etc.)

The novel ECG data collection devices of the present invention feature minimum patient electrodes and leads in conjunction with an integrated Internet-based management service technology that identifies individuals that can benefit from intervention with proven measures to reduce morbidity and mortality rates. The novel ECG data collection devices (apparatus) have highly sensitive ECG leads for ischemia detection using a minimum number of placed electrodes. The apparatus uses a novel form of electrode arrangement to provide lead signals, including the lead II, V₄, V₅, and/or V₆ signals. The apparatus use electrodes positioned on as little as two pads. The first pad has the right arm (RA) electrode and is accordingly placed on the right arm of the subject or, alternatively, on the S position of the torso of the subject. The second pad includes anywhere from two to four electrodes and is placed in the region where the precordial leads V₄, V₅, and V₆ are placed in a standard 12-lead ECG system. Useful ECG data (e.g., lead II and at least one of the V₄, V₅, or V₆ signals) is obtained as long as an electrode on the second pad overlays any one of the V₄, V₅, or V₆ precordial positions on the chest. Very limited training is needed to obtain ECG data using the present invention for two reasons. First, there are only two pads that need to be placed. Second, the devices of the present invention automatically cycle between two or more different lead signals. The information given from these different leads improves the sensitivity of such devices for detecting ischemia. As such, the apparatus of the present invention can be used to acquire highly sensitive or specific ECG data without the assistance of medical training.

Novel methods for screening a population for risk factors associated with coronary heart disease and detection of asymptomatic CHD are made possible because the apparatus of the present invention can be used to obtain highly sensitive ECG data employing medical personnel not specifically trained in conventional ECG procedures. One embodiment of the present invention contemplates a remote hand-held device that can be used to measure ECG and the transport of such data by secured means over the Internet to a server which then analyzes the data to assess the risk level of the subject by finding conditions such as myocardial ischemia and/or silent myocardial infarction (SMI).

One aspect of the present invention provides a computer system for identifying a risk factor for a subject. The computer system comprises a central processing unit and a memory coupled to the central processing unit. The memory stores various instructions, including instructions for receiving data from a remote capture device. The received ECG data is generated for a subject by a device that includes a first non-conductive pad. A first electrode and a second electrode are disposed on the first non-conductive pad and they are adapted for electrical connection with the skin of the subject in order to receive and transmit electrical impulses. The first electrode represents any one of V₄, V₅, or V₆ and the second electrode is either (i) positioned on the subject below (approximately below) the first electrode in order to represent left leg (LL) or (ii) placed on a line on the subject defined by the V₄, V₅, and V₆ precordial positions in order to represent any one of V₄, V₅, or V₆ not represented by the first electrode. As used here, the term approximately below means that the second electrode is placed at some position below the precordial line. In preferred embodiments, the second electrode is positioned at least 12 cM from the heart. Thus, in some embodiments, the second electrode can be positioned anywhere on the patient, including above the first electrode, as long as it is 12 cM away from the heart. In some embodiments, the first electrode represents V₄ or V₅ and is for positioning on V₄ or V₅ of the subject and the second electrode represents LL. The device includes a second non-conductive pad. A third electrode is disposed on the second non-conductive pad and is adapted for electrical connection with the skin in order to receive and transmit electrical impulses. The third electrode is a right arm (RA) electrode that is for positioning on or close to the right arm of the subject. The device includes an electrical connection that connects each electrode disposed on the first and the second non-conductive pad to an electrocardiological measuring apparatus.

The memory further comprises instructions for analyzing and storing the data for the risk factor. In some embodiments, the ECG data that is analyzed is any one or more of providing baseline information, providing evidence of silent myocardial infarction (SMI), or providing evidence of silent and/or inducible ischemia

In some embodiments, the device measures ECG data, which can optionally be digitized. In some cases, the data further comprises results of a blood test (e.g., cholesterol, high density lipoprotein/low density lipoprotein, etc) or other diagnostic tests (e.g., diabetes, etc.) for the subject. In some instances, the data further comprises member personal record information for the subject such as, for example, a name, an address, a telephone number, an age, or an e-mail address, and all risk information associated with the member.

In some embodiments, the data is encrypted. In some instances, the instructions for analyzing the ECG data for the normal/abnormal findings comprises instructions for performing ECG analysis and additional instructions for performing risk factor decision modeling. The instructions for performing ECG analysis determines ECG findings, wherein said ECG findings are an identification of no abnormal ECG findings, or an identification of one or more abnormal ECG findings for the subject. The instructions for decision modeling determine a pre-screening identification for the subject based on a function of the ECG result. In some embodiments, the pre-screening identification is determined by considering risk factor information for the subject. In some embodiments, the risk factors are at least one of an advanced age of the subject (e.g., 50 years or older, 60 years or older, 70 years or older, etc.), hypertension of the subject, elevated serum cholesterol of the subject, cigarette smoking, obesity, physical activity, and diet of the subject, the results from a test for diabetes for the subject, a sex of the subject, family history of the subject or the ethnicity of the subject.

In some embodiments, the memory further comprises a web site module comprising instructions for providing a web site that includes a reporting of the data. In some instances, the web site is secured with a user identification and a password associated with the subject. In some embodiments, the memory further comprises a database having a member record for each subject in a plurality of subjects, where each member record comprises (i) a unique member identifier for the subject corresponding to the member record, (ii) a personal record for the subject corresponding to the member record, and the risk factor information associated with the subject that was obtained by the instructions for receiving data from a remote capture device, and (iii) ECG data for the subject corresponding to the member record that was obtained by the instructions for receiving data from a remote capture device.

In some embodiments, a personal record in the database further comprises results of a blood test (e.g., cholesterol, high density lipoprotein/low density lipoprotein, etc) or other diagnostic tests (e.g., diabetes, etc.) associated with the member record. In some embodiments, the personal record comprises a name, an address, a telephone number, an age, and/or an e-mail address for the subject corresponding to the member record. In some embodiments, a member record in the database further comprises a pre-screening identification for the subject corresponding to the member record.

In still other embodiments, a personal record in the database further comprises risk factors for the subject associated with the member record that comprises an age of the subject, a blood pressure of the subject, a cholesterol level of the subject, the results from a test for diabetes for the subject, a sex of the subject, lifestyle of the subject, and/or the ethnicity of the subject.

### 4. BRIEF DESCRIPTION OF THE DRAWINGS

FIG. 1 illustrates ECG leads I, II, and III in accordance with the prior art.

FIG. 2A illustrates the placement of leads V₁ through V6 on a subject in accordance with the prior art.

FIG. 2B illustrates the cardiac conduction electrocardiographic waveform, in accordance with the known art.

FIG. 2C illustrates the sensitivity of respective leads relative to a 12-lead ECG in a study taken in the known art.

FIG. 3A illustrates an ECG electrode pad in which the six precordial electrodes (V₁ through V₆) are plated and etched on a flexible adhesive pad in a pattern designed to place these electrodes at the correct precordial positions in accordance with the prior art.

FIG. 3B illustrates a conventional bipolar three lead monitoring system in accordance with the prior art.

FIG. 3C illustrates a conventional five lead unipolar monitoring system in accordance with the prior art.

FIG. 3D illustrates seven lead ambulatory ECG electrode configuration in accordance with the prior art.

FIG. 3E illustrates a modified V₅ system that is used by anesthesiologists during surgery or by cardiologists for ambulatory ECG monitoring in accordance with the prior art.

FIG. 4 illustrates commonly used bipolar V₅ configurations in accordance with the prior art.

FIG. 5 illustrates an electrode configuration in accordance with one embodiment of the present invention.

FIG. 6 illustrates an electrocardiological measuring device for automatic ECG-lead switch control in accordance with one embodiment of the present invention.

FIG. 7 illustrates how an electrocardiological measuring device for automatic ECG-lead switch control measures different signals during discrete time intervals in accordance with one embodiment of the present invention.

FIG. 8 illustrates a three-electrode configuration in accordance with one embodiment of the present invention.

FIG. 9 illustrates a four-electrode configuration in accordance with an embodiment of the present invention.

FIG. 10 illustrates a four-electrode configuration in accordance with another embodiment of the present invention.

FIG. 11A illustrates a five-electrode configuration in accordance with another embodiment of the present invention.

FIG. 11B illustrates a two-electrode configuration in accordance with another embodiment of the present invention.

FIG. 11C illustrates additional electrode configurations in accordance with the present invention.

FIG. 12 illustrates a system for screening a general population for risk factors associated with coronary heart disease in accordance with one embodiment of the present invention.

FIG. 13 illustrates the structure of a database in accordance with one embodiment of the present invention.

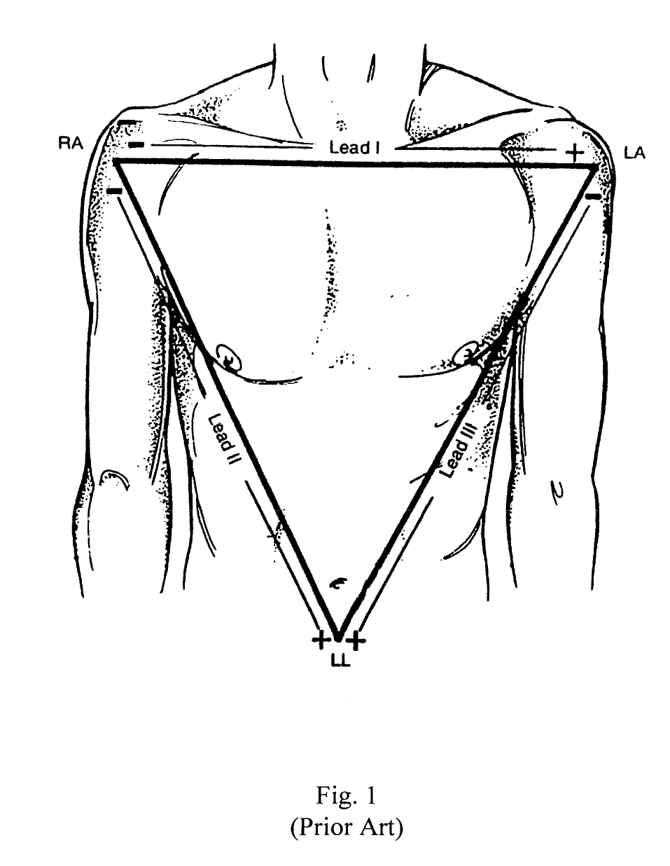

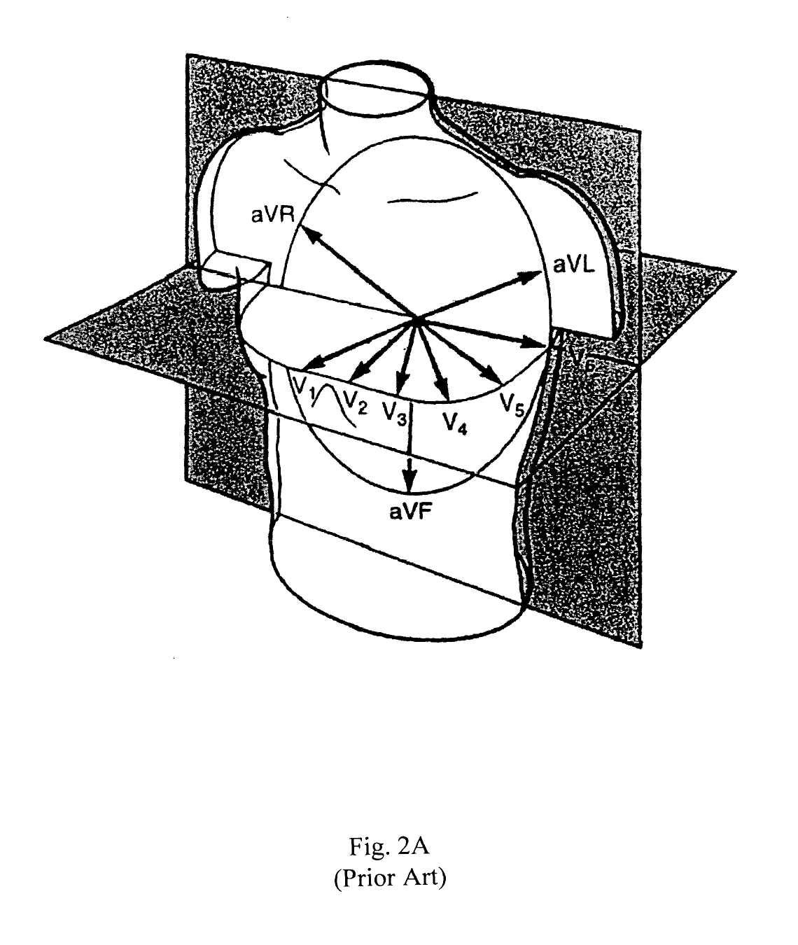

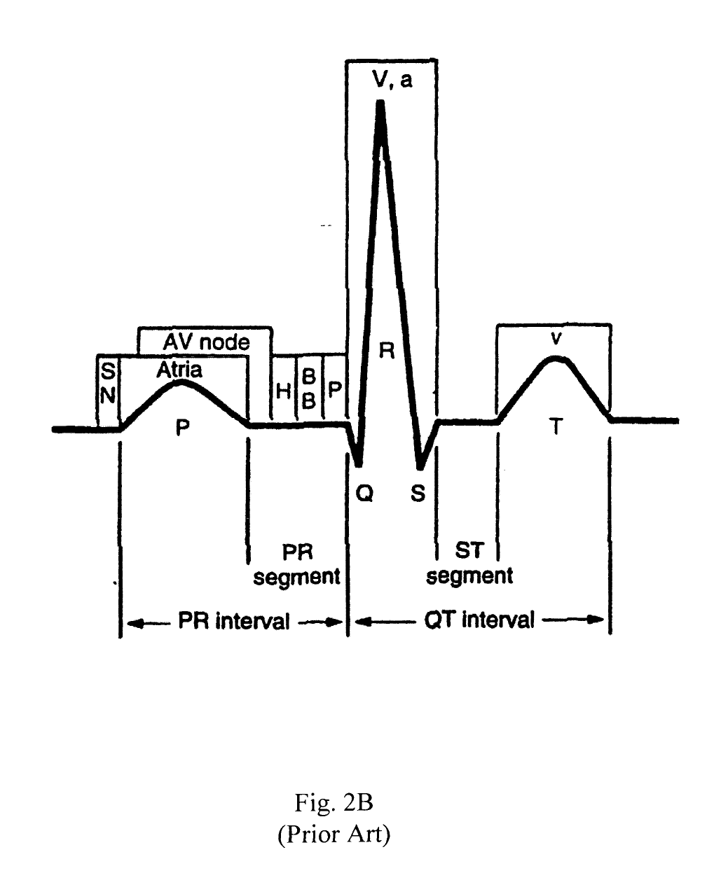

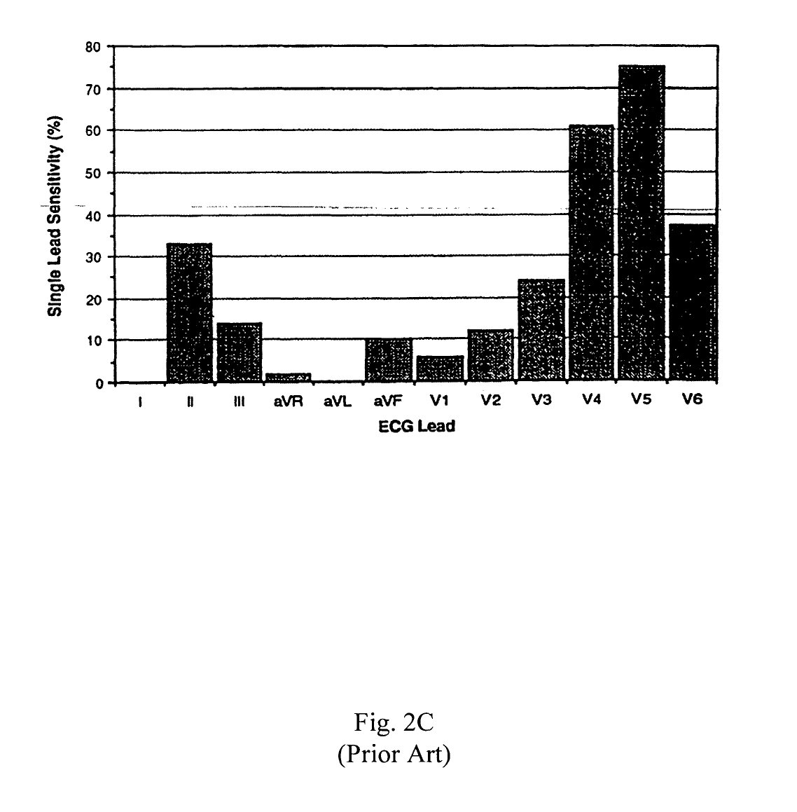

Like reference numerals refer to corresponding parts throughout the several views of the drawings.

### 5. DETAILED DESCRIPTION OF THE INVENTION

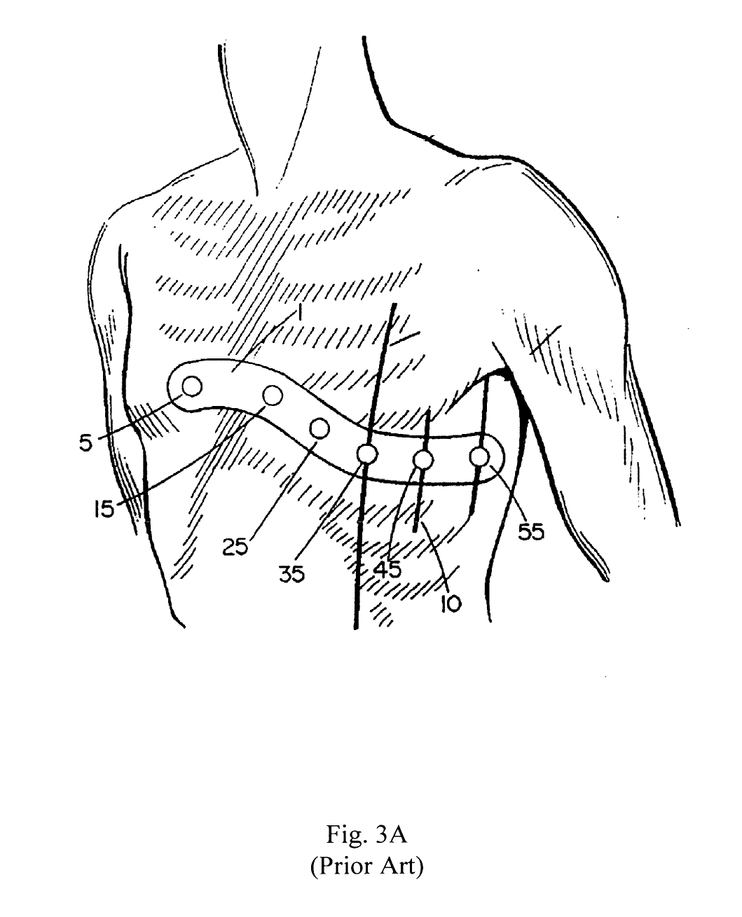

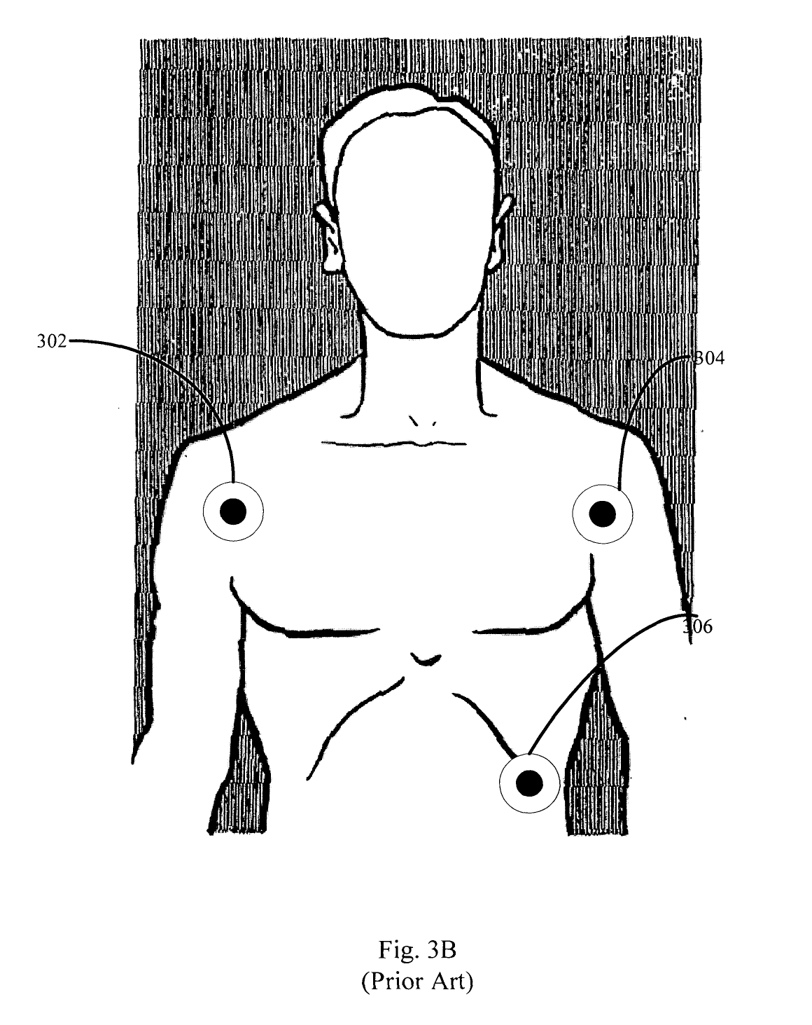

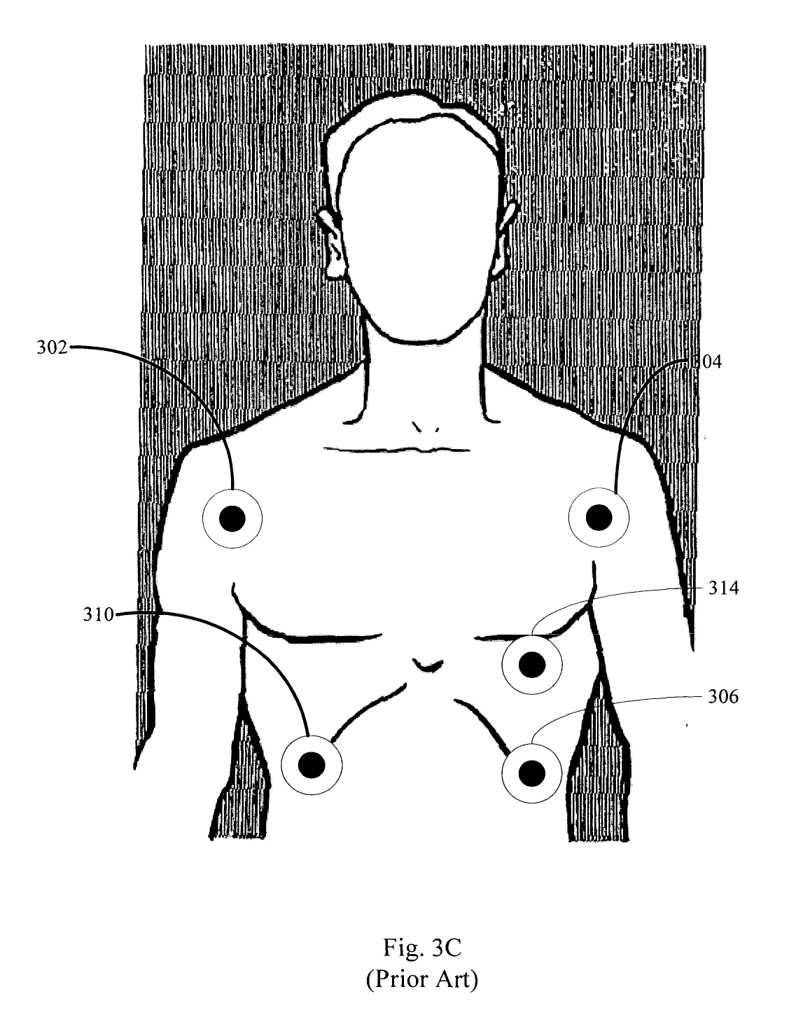

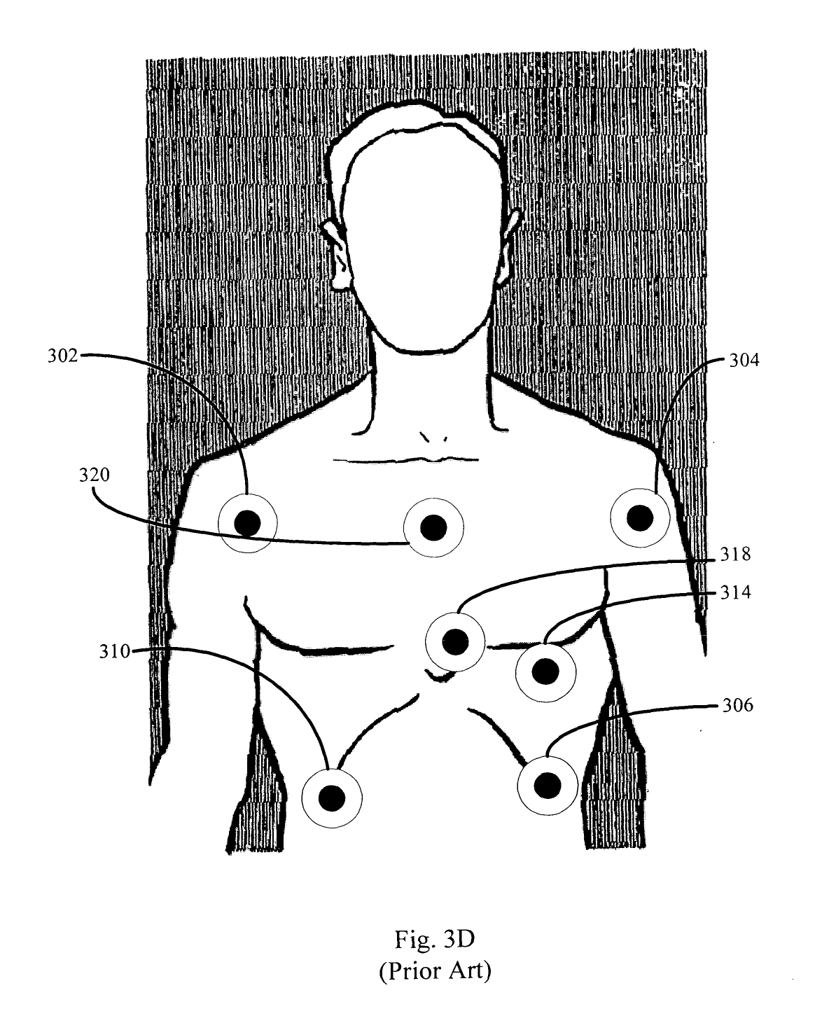

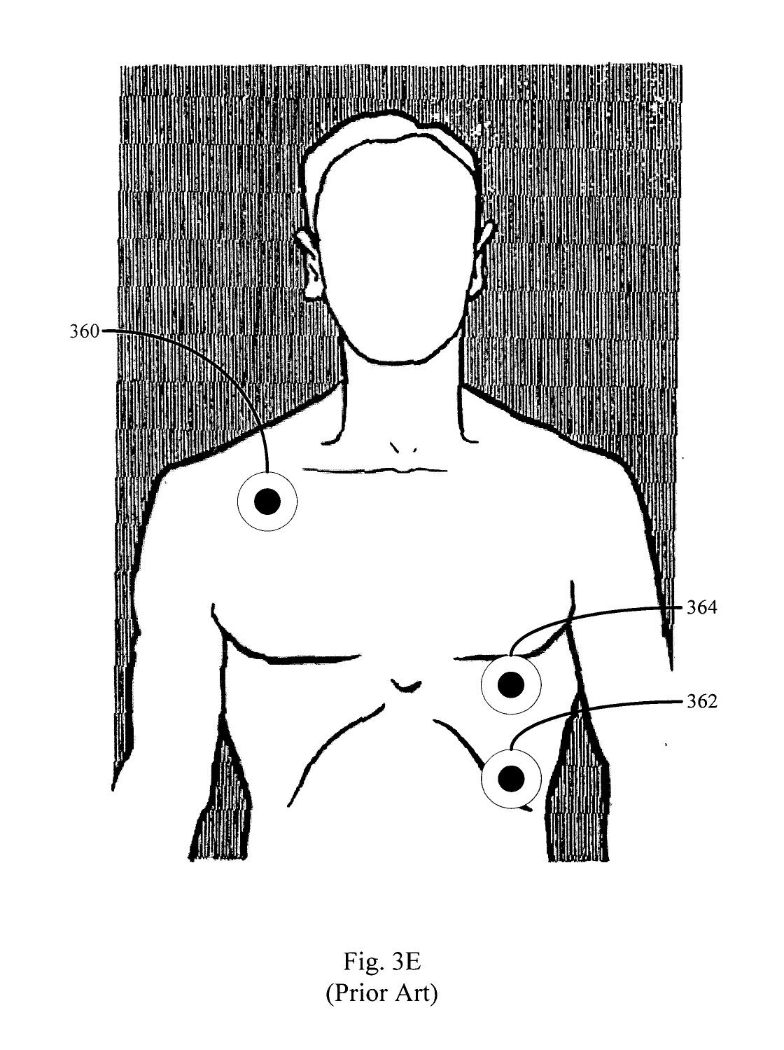

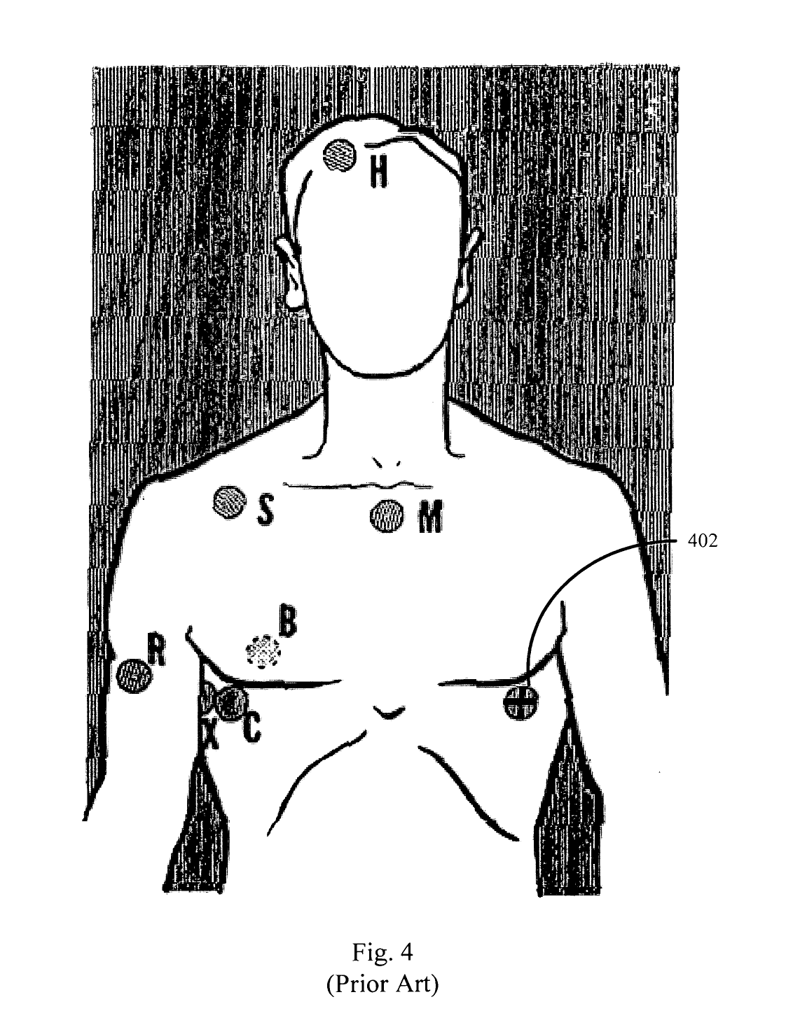

The present invention provides apparatus and methods for obtaining sensitive ECG data using a reduced electrode set. The apparatus and methods of the present invention use an adaptation of the bipolar lead known as the modified V₅ lead. See London and Kaplan, “Advances in electrocardiographic monitoring” in Kaplan, 3^{rd} edition, 1993, Cardiac Anesthesia, Philadelphia, W B Saunders, p. 323, which is hereby incorporated by reference in its entirety, for a description of such bipolar leads. See also Section 2.8, above, and FIG. 3E. FIG. 4 illustrates a number of bipolar V₅ configurations, including a CS₅ lead, commonly referred to as modified V₅. To achieve a bipolar lead, the positive electrode is placed on the precordial V₅ location 402. The negative electrode is placed at any of the locations marked by upper case letters in FIG. 4, which by convention are the second prefix of the lead (the first is “C”). See London and Kaplan, Id. The CS₅ lead is obtained by placing the RA electrode at the S position and placing the LA electrode at the precordial V₅ position. The modified V₅ lead is closest to the CS₅ lead. In the modified V₅ lead, the RA lead is moved to a position close to the normal right arm position (near the S position) and placing the LA lead on the precordial V₅ position. By selecting lead I on a three-lead monitor, this lead data is obtained. See Froelicher, 1987, Exercise and the Heart, Chicago, Year Book Medical Publishers, Inc., p. 20.

### 5.1 Lead II and V4(V5) From Three Electrodes

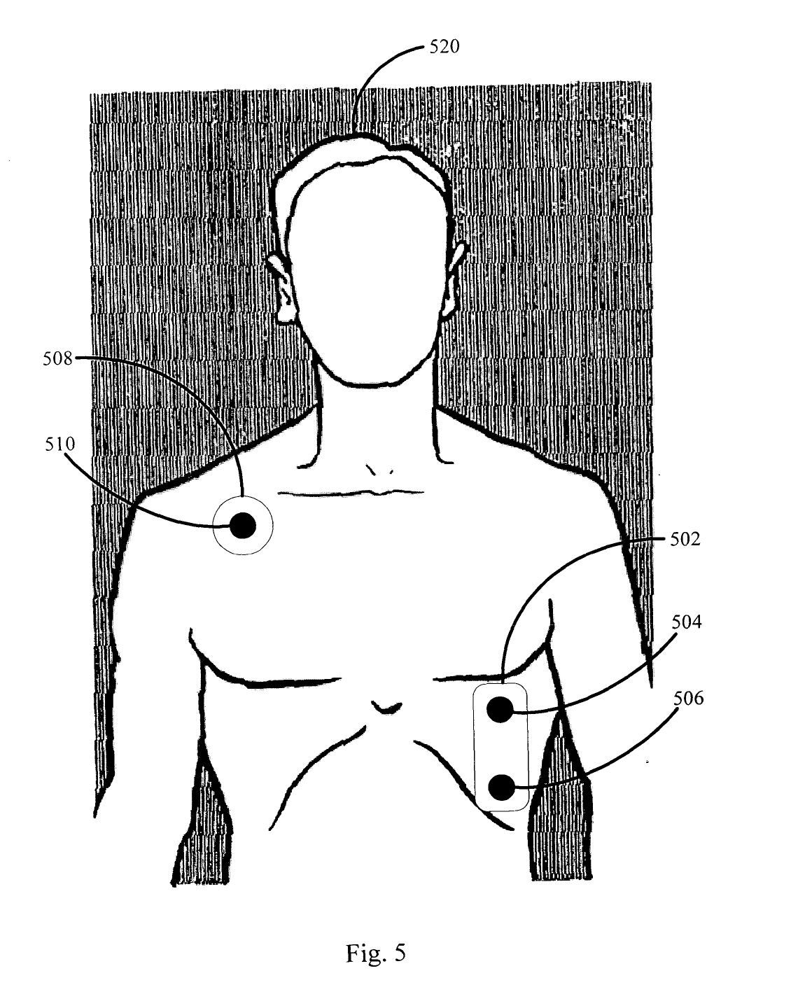

FIG. 5 illustrates an ECG electrode configuration in accordance with one embodiment of the present invention. In the electrode configuration, a first non-conductive pad 502 and a second non-conductive pad 508 are used. A first electrode 504 and a second electrode 506 are disposed on pad 502. Electrodes 504 and 506 are adapted for electrical connection with the skin of a subject 520 in order to receive and transmit electrical impulses. In the embodiment illustrated in FIG. 5, electrode 504 is placed in the precordial V₄ or V₅ position. Electrode 506 represents left leg (LL) and is positioned on subject 520 below electrode 504. A third electrode 510 is disposed on the second non-conductive pad 508. Electrode 510 is adapted for electrical connection with the skin in order to receive and transmit electrical impulses. Electrode 510 is a right arm (RA) electrode that is for positioning at a position that is on or close to the right arm of subject 520. In some embodiments, electrode 510 is placed on or near the S position depicted in FIG. 4.

Although not shown, an apparatus in accordance with the embodiment of the present invention illustrated in FIG. 5 includes an electrical connection that connects each electrode disposed on pads 502 and 508 to an electrocardiological measuring apparatus. The two pad electrode system illustrated in FIG. 5 is highly advantageous because only two pads need to be positioned on the body. If pad 502 is placed such that electrode 504 overlies the V₄ precordial position (position 35, FIG. 3), then the V₄ signal is measured by the electrocardiological measuring apparatus. If, on the other hand, pad 502 is placed such that electrode 504 overlays the V₅ precordial position (position 45, FIG. 3), then the V₅ signal is measured by the electrocardiological measuring apparatus. The electrode configuration illustrated in FIG. 5 can also be used to measure lead II from electrode 506 (positive) to electrode 510 (negative) when electrode 504 is held as ground.

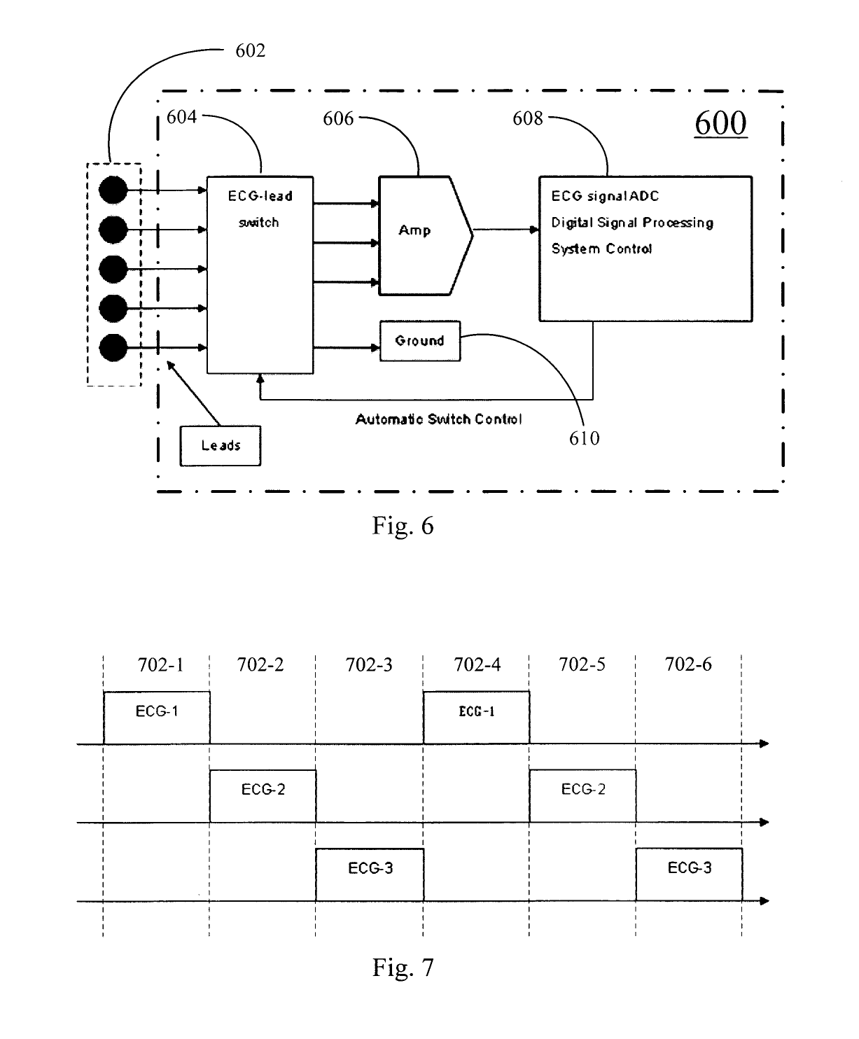

Advantageously, the electrode configuration illustrated in FIG. 5 can be used to automatically measure both (i) the V₄ or V₅ signal and (ii) lead II using the electrocardiological measuring device 600 illustrated in FIG. 6. Device 600 receives input from electrodes 602 (e.g., the electrodes 504, 506, and 510 of FIG. 5). Lead selection (e.g., V₄ signal, V₅, signal, lead II signal) is determined by ECG-lead switch 604. ECG-lead switch 604 is directed to generate a predetermined lead by digital signal processing system control 608. ECG-lead switch 604 generates a given lead by assigning one or more specific electrodes as “positive”, one or more specific electrodes as “negative”, and one or more specific electrodes as “ground”. For example, in the case of the electrode configuration illustrated in FIG. 5, control 608 can request a V₄ or a V₅ signal. When such a request is made, ECG-lead switch 604 assigns electrode 504 as the positive electrode, electrode 510 as the negative electrode, and electrode 506 as ground.

Multiple leads are collected by control 608 by periodically or sequentially making a lead selection request to ECG-lead switch 604 as illustrated in FIG. 7. For example, at time 702-1, control 608 can request ECG signal 1 (ECG-1). In the case of the embodiment illustrated in FIG. 5, ECG-1 could be the V₄ (or V₅) signal. Then, at time 702-2, control 608 can request the lead II signal. In FIG. 7, control 608 requests a third type of signal at time 702-3. However, the number of different signals (e.g., the V₁, V₂, V₃, V₄, V₅, V₆ signals, leads I, II, and III, or other signal forms) that are requested by control 608 will depend upon the number of different possible signals that can be measured by a given electrode configuration. For example, in the electrode configuration illustrated in FIG. 5, two different signals are measured. Accordingly, given the electrode configuration illustrated in FIG. 5, during any given time period 702 (FIG. 7), control 608 will request either the V₄ (V₅) signal or lead II. In other electrode configurations disclosed in the following sections below, more than two signals are measured by control 608 using switch 604. In typical embodiments, control 608 rotates through each of the possible signals that can be measured by a given electrode configuration (e.g., the electrode configuration illustrated in FIG. 5) in a round robin style in which a specific signal is measured during each time period 702. However, more complex sampling patterns are possible. For example, in some embodiments it can be advantageous to measure one signal for a period that is twice as long as a another signal. Accordingly, control 608 can be programmed to collect a first signal (e.g., V₄, V₅, lead II, etc.) for multiple time intervals 702 before collecting a second signal. Communication of signals between switch 604 and control 608 is facilitated by amplifier 606 and the use of ground 610.

A system in accordance with one embodiment of the present invention has now been disclosed. The system allows for the measurement of multiple leads while requiring the placement of only two pads. The system automatically cycles between the available leads in order to improve sensitivity in detecting ischemia. Therefore, less technical expertise is required to use the disclosed system for two reasons: (i) simpler electrode design (two pads) and (ii) simpler lead collections (automatic cycling between available leads. In one example, the system cycles between the lead II and V₅ signal, thereby achieving an estimated sensitivity of eighty percent relative to a 12-lead ECG system.

### 5.2 V₄ and V₅ From Three Electrodes

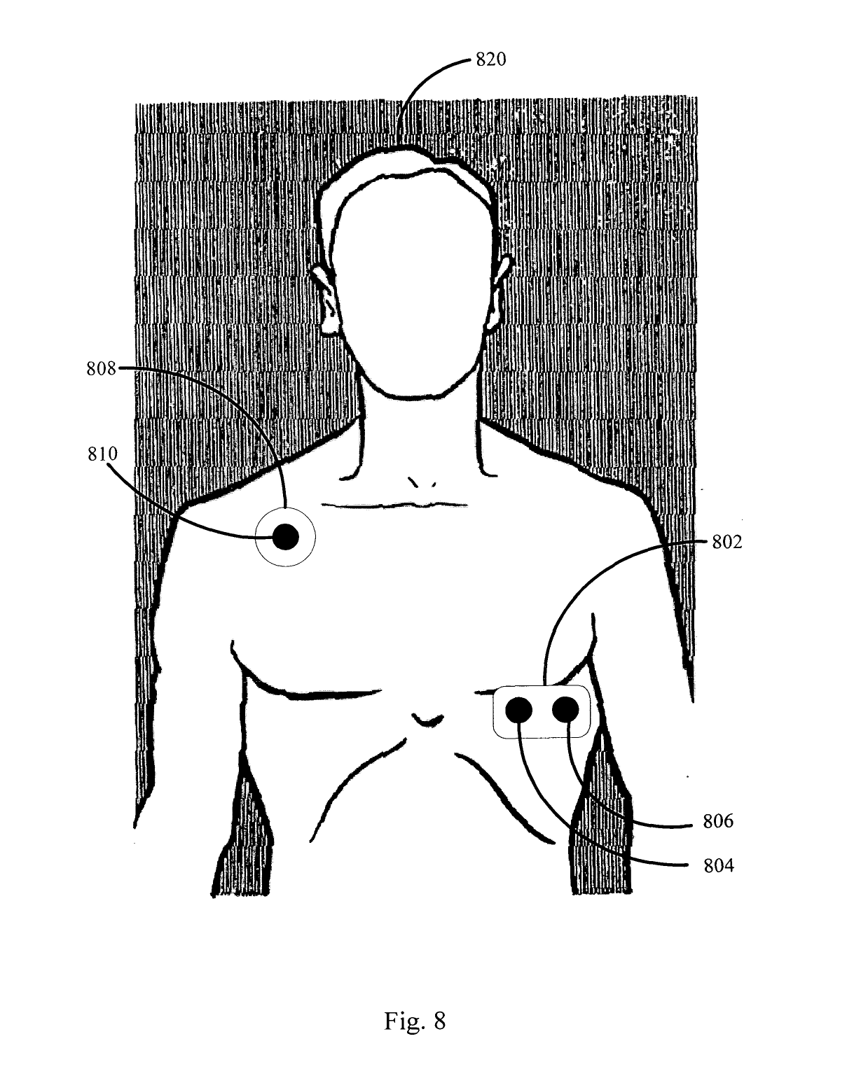

FIG. 8 illustrates the use of an electrode configuration in accordance with another embodiment of the present invention. The electrode configuration includes a first non-conductive pad 802 and a second non-conductive pad 808. A first electrode 804 and a second electrode 806 are disposed on pad 802. Electrodes 804 and 806 are adapted for electrical connection with the skin of a subject 820 in order to receive and transmit electrical impulses. In the embodiment illustrated in FIG. 8, electrode 804 is placed in the precordial V₄ position and electrode 806 is placed in the precordial V₅ position. A third electrode 810 is disposed on the second non-conductive pad 808. Electrode 810 is adapted for electrical connection with the skin in order to receive and transmit electrical impulses. Electrode 810 is a right arm (RA) electrode that is for positioning at a position that is on or close to the right arm of subject 820. In some embodiments, electrode 810 is placed on or near the S position depicted in FIG. 4.

Although not shown, an apparatus in accordance with the embodiment of the present invention illustrated in FIG. 8 includes an electrical connection that connects each electrode disposed on pads 802 and 808 to an electrocardiological measuring apparatus, such as device 600 (FIG. 6). The electrode system illustrated in FIG. 8 is highly advantageous because only two pads must be placed on subject 820. In the case of the electrode configuration illustrated in FIG. 8, either electrode 804 or 806 can be placed over the more sensitive V₅ position in order to collect the V₅ signal. It is contemplated that the V₅ signal is more desirable because a study of such signals found that lead V₅ has the greatest sensitivity at detecting ischemic ST-segment changes (75%) followed by lead V₄ (61%). In contrast, leads V₃ and V₆ are significantly less sensitive, with sensitivities of approximately 23% and 38%, respectively. See London et al., 1988, Anesthesiology 69, p. 232-241.

When electrodes 804 and 806 are respectively positioned on the precordial V₄ and V₅ positions, both the V₄ and V₅ signals can be measured using the intermittent scheme described in Section 5.1, in conjunction with FIGS. 6 and 7. Specifically, at some time intervals 702, the V₄ signal is collected between electrode 810 and 804 with electrode 806 serving as ground while at other time intervals 702, the V₅ signal is collected between electrode 810 and 806 with electrode 804 serving as ground. When electrode 804 is positioned on the precordial position V₅, only the V₅ signal is measured using electrode 804 as the positive terminal with electrode 806 as ground. When electrode 806 is positioned on the V₄ position, only the V₄ signal is measured using electrode 806 as the positive terminal with 804 as ground.

Another system in accordance with one embodiment of the present invention has now been disclosed. The system allows for the measurement of multiple leads while requiring the placement of only two pads. The system automatically cycles between the available leads in order to improve sensitivity in detecting ischemia. Therefore, less technical expertise is required to use the disclosed system for two reasons: (i) simpler electrode design (two pads) and (ii) simpler lead collections (automatic cycling between available leads. In one example, the system cycles between the V₄ and V₅ signal, thereby achieving an estimated sensitivity of ninety percent relative to a 12-lead ECG system.

### 5.3 Four Lead ECG

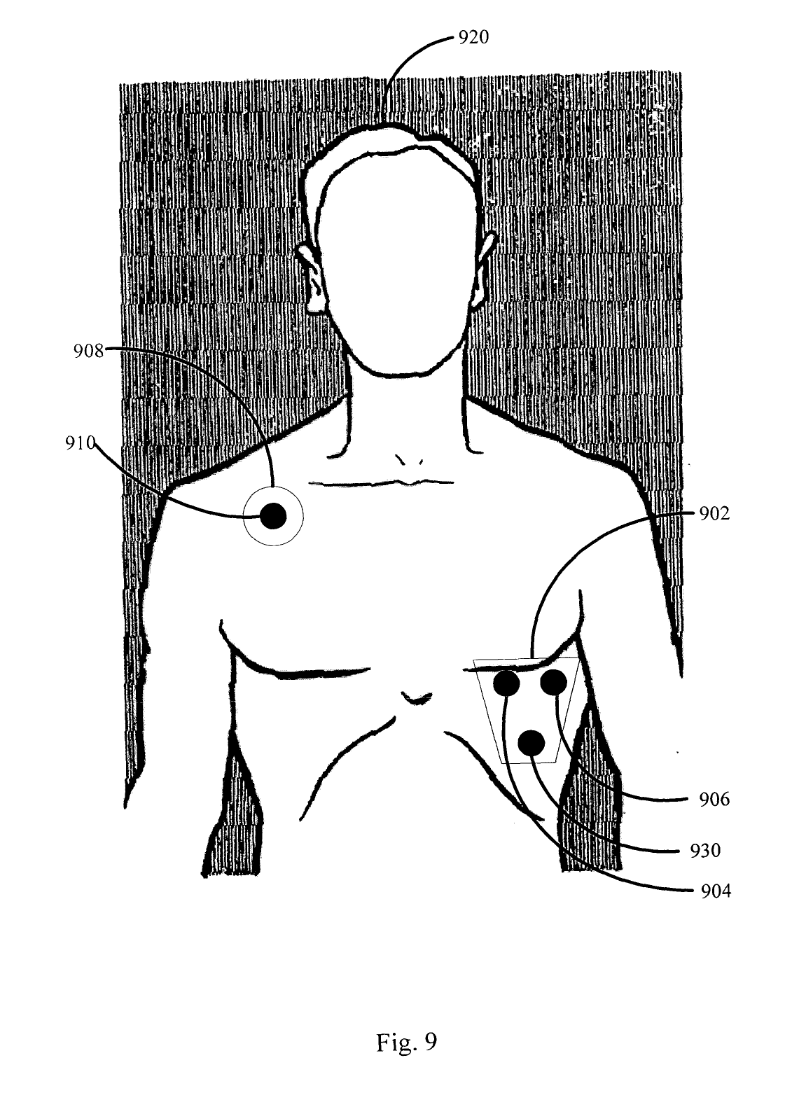

FIG. 9 illustrates an electrode configuration in accordance with another embodiment of the present invention. The electrode configuration includes a first non-conductive pad 902 and a second non-conductive pad 908. Electrodes 904, 906, and 930 are disposed on pad 902. Electrodes 904, 906, and 930 are adapted for electrical connection with the skin of a subject 920 in order to receive and transmit electrical impulses. In the embodiment illustrated in FIG. 9, electrode 904 is optimally placed in the precordial V₄ position and electrode 906 is optimally placed in the precordial V₅ position. Electrode 910 is disposed on pad 908. Electrode 910 is adapted for electrical connection with the skin in order to receive and transmit electrical impulses. Electrode 910 is a right arm (RA) electrode that is for positioning at a position that is on or close to the right arm of subject 920. In some embodiments, electrode 910 is placed on or near the S position depicted in FIG. 4.

Although not shown, an apparatus in accordance with the embodiment of the present invention illustrated in FIG. 9 includes an electrical connection that connects each electrode disposed on pads 902 and 908 to an electrocardiological measuring apparatus, such as device 600 (FIG. 6). The electrode system illustrated in FIG. 9 is highly advantageous because only two pads must be positioned.

Tables 2 through 6 illustrate various methods by which the electrode configuration illustrated in FIG. 9 can be used to measure select combinations of the V₄ signal, the V₅ signal, and lead II.

TABLE 2
Lead II and V₄ signal, first alternative

| | Positive electrode | Negative electrode | Ground electrode |
|---|---|---|---|
| Lead II | 930 | 910 | 906 |
| V₄ signal | 904 | 910 | 906 |

TABLE 3
Lead II and V₄ signal, second alternative

| | Positive electrode | Negative electrode | Ground electrode |
|---|---|---|---|
| Lead II | 930 | 910 | 906 |
| V₄ signal | 904 | aVL (from 908 and | 906 |
| | | 930) | |

TABLE 4
Lead II and V₅ signal, first alternative

| | Positive electrode | Negative electrode | Ground electrode |
|---|---|---|---|
| Lead II | 930 | 910 | 904 |
| V₅ signal | 906 | 910 | 904 |

TABLE 5
Lead II and V₅ signal, second alternative

| | Positive electrode | Negative electrode | Ground electrode |
|---|---|---|---|
| Lead II | 930 | 910 | 904 |
| V₅ signal | 906 | aVL (from 910 and | 904 |
| | | 930) | |

TABLE 6
V₄ and V₅ signal

| | Positive electrode | Negative electrode | Ground electrode |
|---|---|---|---|
| V₄ signal | 904 | 910 | 930 |
| V₅ signal | 906 | 910 | 930 |

When electrodes 904 and 906 are respectively positioned on the precordial V₄ and V₅ positions, the V₄ and V₅ signals are measured using any of the connections in Tables 2 through 6 and the intermittent scheme described in Section 5.1, in conjunction with FIGS. 6 and 7. For example, consider the case in which lead II and the V₄ signal are measured in accordance with Table 2. At some time period 702, control 608 instructs switch 604 to measure lead II. In response, switch 604 sets electrode 930 positive, electrode 910 negative, and electrode 906 to ground and collects lead II for a predetermined period of time (e.g., five seconds, 30 seconds, 1 minute, between five seconds and five minutes, more than five minutes, etc.). Then, at another time period 702, control 608 instructs switch 604 to measure the V₄ signal. In response, switch 604 sets electrode 904 positive, electrode 910 negative, and electrode 906 to ground for a predetermined period of time. In this way, the same minimal set of leads can be used to collect multiple signals (e.g., V₄, V₅, lead II) using the same electrode configuration. Any of the signals listed in Tables 2 through 6 can be measured by electrocardiological apparatus 600 during discrete time intervals 702.

A second possible outcome is that electrode 904 is positioned on the precordial position V₅ rather than on position V₄. In such instances, the V₅ and lead II signals can be measured using the electrode configurations specified in Table 7.

TABLE 7
Electrode 904 incorrectly positioned on precordial V₅ position

| | Positive electrode | Negative electrode | Ground electrode |
|---|---|---|---|
| Lead II | 930 | 910 | 904 or 906 |
| V₅ signal | 904 | 910 | 930 |

A third possible outcome is that electrode 906 is positioned on the precordial V₄ position rather than on position V₅. In such instances, the V₄ and lead II signals are measured using the electrode configurations specified in Table 8.

TABLE 8
Electrode 906 incorrectly positioned on precordial V₄ position

| | Positive electrode | Negative electrode | Ground electrode |
|---|---|---|---|
| Lead II | 930 | 910 | 904 or 906 |
| V₄ signal | 906 | 910 | 930 |

A third system in accordance with one embodiment of the present invention has now been disclosed. The system allows for the measurement of multiple leads while requiring the placement of only two pads. The system automatically cycles between the available leads in order to improve sensitivity in detecting ischemia. Therefore, less technical expertise is required to use the disclosed system for two reasons: (i) simpler electrode design (two pads) and (ii) simpler lead collections (automatic cycling between available leads. In one example, the system cycles between the V₄, V₅, and the Lead II signals, thereby achieving an estimated sensitivity of 96 percent relative to a 12-lead ECG system.

### 5.4 Additional Four Lead ECG Embodiment

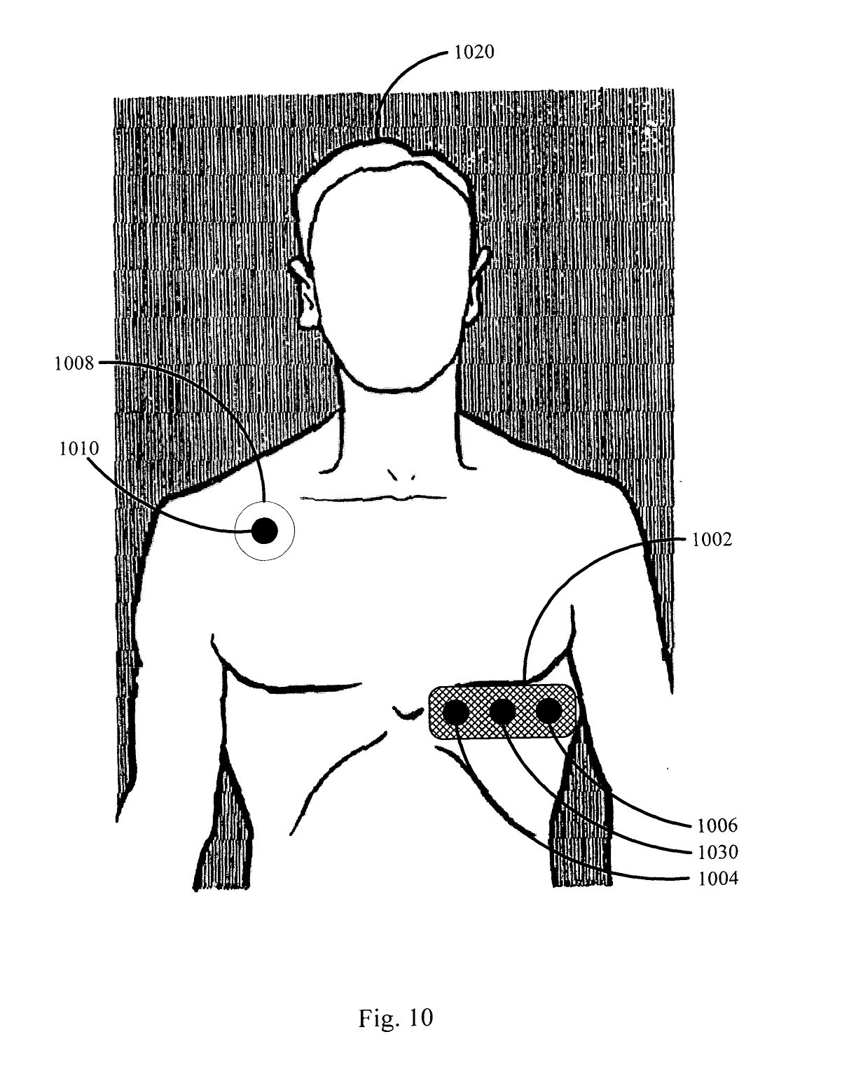

FIG. 10 illustrates an electrode configuration in accordance with another embodiment of the present invention. The electrode configuration includes a first non-conductive pad 1002 and a second non-conductive pad 1008. Electrodes 1004, 1006, and 1030 are disposed on pad 1002. Electrodes 1004, 1006, and 1030 are adapted for electrical connection with the skin of a subject 1020 in order to receive and transmit electrical impulses. In the embodiment illustrated in FIG. 10, electrode 1004 is optimally placed in the precordial V₄ position, electrode 1030 is optimally placed in the precordial V₅ position, and electrode 1006 is optimally placed in the precordial V₆ position. Electrode 1010 is disposed on pad 1008. Electrode 1010 is adapted for electrical connection with the skin in order to receive and transmit electrical impulses. Electrode 1010 is a right arm (RA) electrode that is for positioning at a position that is on or close to the right arm of subject 1020. In some embodiments, electrode 1010 is placed on or near the S position of the subject, where the S position is as depicted in FIG. 4.

Although not shown, an apparatus that can use the electrical configuration illustrated in FIG. 10 connects each electrode disposed on pads 1002 and 1008 to an electrocardiological measuring apparatus, such as device 600 (FIG. 6). The electrode system illustrated in FIG. 10 is advantageous because only two pads are positioned on a subject and the pads can be correctly positioned without the help of medically trained professionals. Tables 9 through 11 illustrate various methods by which the electrode configuration illustrated in FIG. 10 can be used to measure select combinations of the V₄ signal, the V₅ signal, and the V₆ signal, where electrode 1004 is placed in position V₄, electrode 1030 is placed in position V₅, and electrode 1006 is placed in position V₆.

TABLE 9
V₄ and V₅ signals

| | Positive electrode | Negative electrode | Ground electrode |
|---|---|---|---|
| V₄ signal | 1004 | 1010 | 1006 |
| V₅ signal | 1030 | 1010 | 1006 |

TABLE 10
V₄ and V₆ signals

| | Positive electrode | Negative electrode | Ground electrode |
|---|---|---|---|
| V₄ signal | 1004 | 1010 | 1030 |
| V₆ signal | 1006 | 1010 | 1030 |

TABLE 11
V₅ and V₆ signals

| | Positive electrode | Negative electrode | Ground electrode |
|---|---|---|---|
| V₅ signal | 1030 | 1010 | 1004 |
| V₆ signal | 1006 | 1010 | 1004 |

In preferred embodiments, electrodes 1004, 1030, and 1006 are respectively positioned on the precordial V₄, V₅, and V₆ positions. In such instances, the V₄, V₅ and V₆ signals are measured using any of the connections described in Tables 9 through 11 and the intermittent scheme described in Section 5.1, in conjunction with FIGS. 6 and 7.

A second possible outcome is that electrodes 1030 and 1006 are respectively positioned on the precordial V₄ and V₅ positions. In such instances, the V₄ and V₅ signals can be measured using the electrode configurations specified in Table 12.

TABLE 12
Electrodes 1030 and 1006 incorrectly positioned on the V₄ and V₅
positions, respectfully

| | Positive electrode | Negative electrode | Ground electrode |
|---|---|---|---|
| V₄ signal | 1030 | 1010 | 1006 |
| V₅ signal | 1006 | 1010 | 1004 |

A third possible outcome is that electrodes 1004 and 1030 are respectively positioned on the precordial V₅ and V₆ positions. In such instances, the V₅ and V₆ signals are measured using the electrode configurations specified in Table 13.

TABLE 13
Electrodes 1004 and 1030 incorrectly positioned on the V₅ and V₆
positions, respectfully

| | Positive electrode | Negative electrode | Ground electrode |
|---|---|---|---|
| V₅ signal | 1004 | 1010 | 1006 |
| V₆ signal | 1030 | 1010 | 1004 |

### 5.5 Additional Electrode Configurations

In addition to the systems that include the electrode configurations described in Sections 5.1 through 5.4, above, the present invention provides systems that provide additional electrode configurations that are described in the following subsections. When the electrode configurations provide the possibility of measuring more than one lead, the present invention supports the automatic sampling of these multiple leads, thereby increasing the sensitivity of the devices without increasing the skills required by the user. As such, the systems described in the following subsections, in addition to the systems described in Sections 5.1 through 5.4, above, provide for the ability to perform high sensitivity self-screening.

### 5.5.1 Five lead ECG

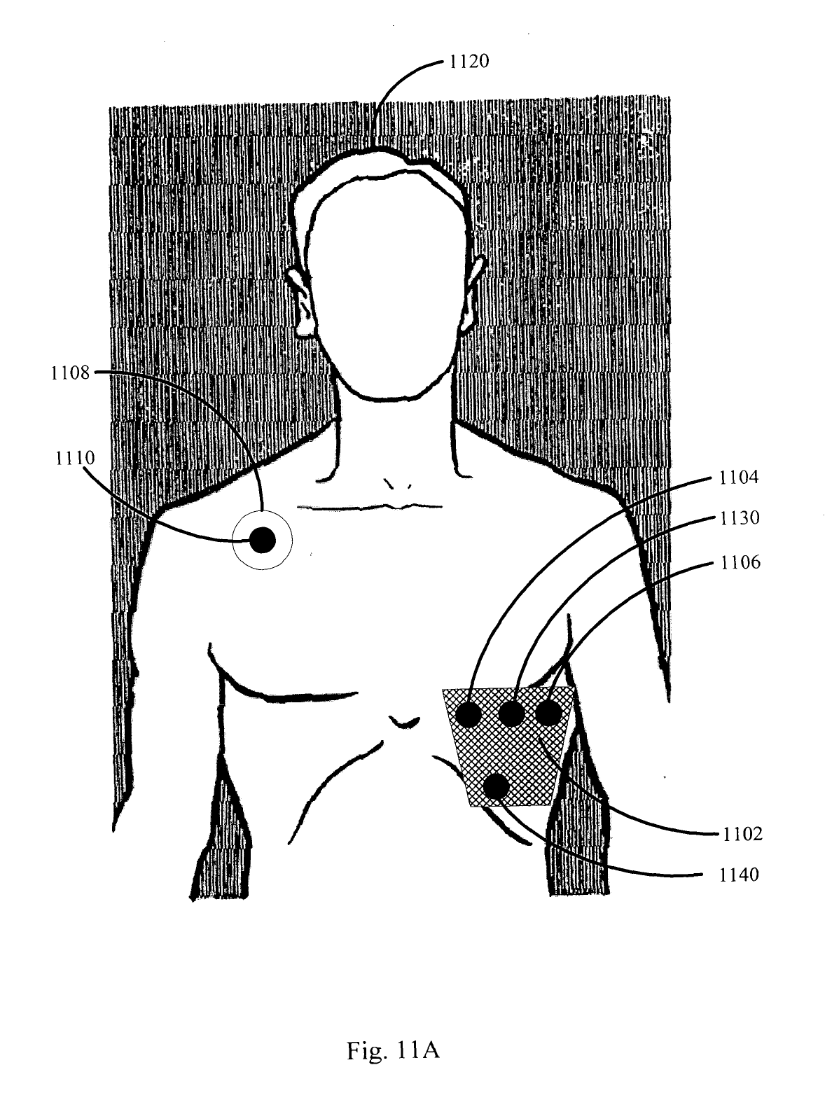

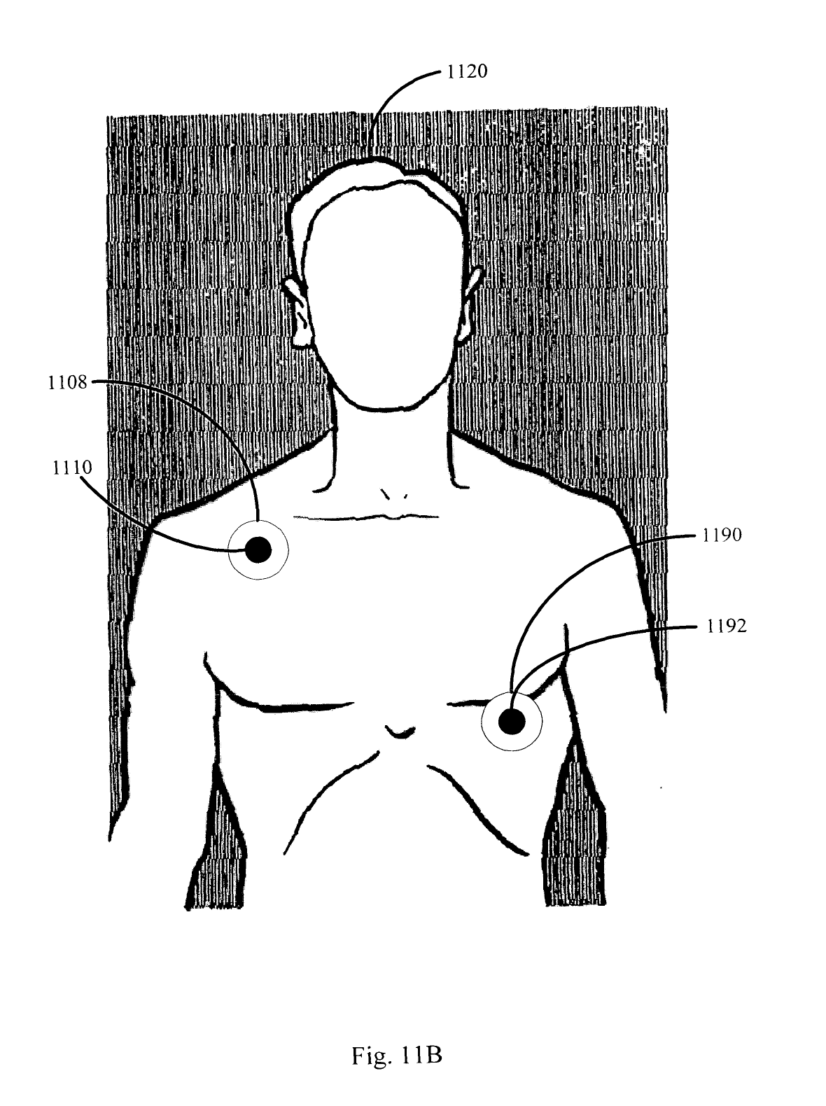

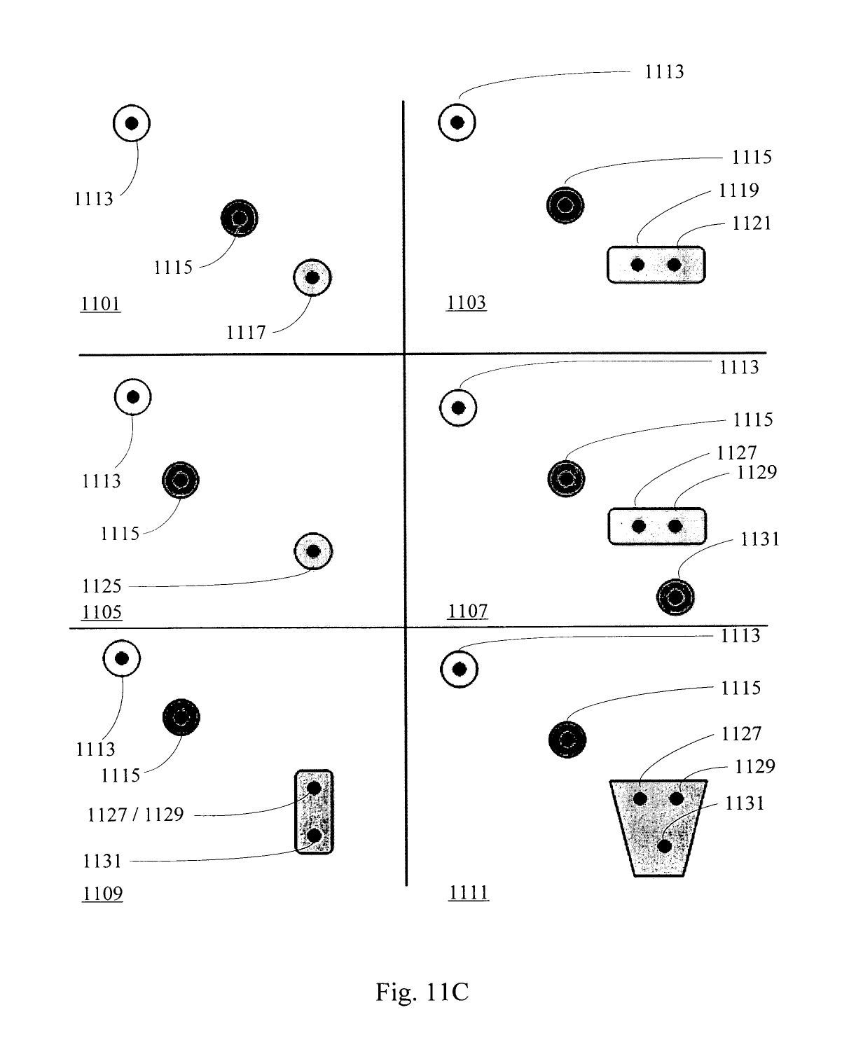

FIG. 11A illustrates an electrode configuration in accordance with another embodiment of the present invention. The electrode configuration includes a first non-conductive pad 1102 and a second non-conductive pad 1108. Electrodes 1104, 1106, 1130 and 1140 are disposed on pad 1102. These electrodes are adapted for electrical connection with the skin of a subject 1120 in order to receive and transmit electrical impulses. In the embodiment illustrated in FIG. 11A, electrode 1104 is optimally placed in the precordial V₄ position, electrode 1130 is optimally placed in the precordial V₅ position, and electrode 1106 is optimally placed in the precordial V₆ position. Electrode 1140 serves as ground. Electrode 1110 is disposed on pad 1108. Electrode 1110 is adapted for electrical connection with the skin in order to receive and transmit electrical impulses. Electrode 1110 is a right arm (RA) electrode that is for positioning at a position that is on or close to the right arm of subject 1020. In some embodiments, electrode 1110 is placed on or near the S position of the subject, where the S position is as depicted in FIG. 4.

Although not shown, an apparatus that can use the electrical configuration illustrated in FIG. 11A connects each electrode disposed on pads 1102 and 1108 to an electrocardiological measuring apparatus, such as device 600 (FIG. 6). The electrode system illustrated in FIG. 11A is highly advantageous because only two pads must be correctly positioned on a subject and this can be accomplished without the help of medically trained professionals. Tables 14 through 20 illustrate various methods by which the electrode configuration illustrated in FIG. 11A can be used to measure select combinations of the V₄ signal, the V₅ signal, the V₆ signal and lead II.

TABLE 14
Lead II and the V₄ and V₅ signals, first alternative

| | Positive electrode | Negative electrode | Ground electrode |
|---|---|---|---|
| V₄ signal | 1104 | 1110 | 1106 |
| V₅ signal | 1130 | 1110 | 1106 |
| Lead II | 1140 | 1110 | 1106 |

TABLE 15
Lead II and the V₄ and V₅ signals, second alternative

| | Positive electrode | Negative electrode | Ground electrode |
|---|---|---|---|
| V₄ signal | 1104 | aVL (from 1110 | 1106 |
| | | and 1140) | |
| V₅ signal | 1130 | aVL (from 1110 | 1106 |
| | | and 1140) | |
| Lead II | 1140 | 1110 | 1106 |

TABLE 16
V₄, V₆ signals and lead II, first alternative

| | Positive electrode | Negative electrode | Ground electrode |
|---|---|---|---|
| V₄ signal | 1104 | 1110 | 1130 |
| V₆ signal | 1106 | 1110 | 1130 |
| Lead II | 1140 | 1110 | 1130 |

TABLE 17
V₄, V₆ signals and lead II, second alternative

| | Positive electrode | Negative electrode | Ground electrode |
|---|---|---|---|
| V₄ signal | 1104 | aVL (from 1110 | 1130 |
| | | and 1140) | |
| V₆ signal | 1106 | aVL (from 1110 | 1130 |
| | | and 1140) | |
| Lead II | 1140 | 1110 | 1130 |

TABLE 18
V₅, V₆ signals and lead II, first alternative

| | Positive electrode | Negative electrode | Ground electrode |
|---|---|---|---|
| V₅ signal | 1130 | 1110 | 1104 |
| V₆ signal | 1106 | 1110 | 1104 |
| Lead II | 1140 | 1110 | 1104 |

TABLE 19
V₅, V₆ signals and lead II, second alternative

| | Positive electrode | Negative electrode | Ground electrode |
|---|---|---|---|
| V₅ signal | 1130 | aVL (from 1110 | 1104 |
| | | and 1140) | |
| V₆ signal | 1106 | aVL (from 1110 | 1104 |
| | | and 1140) | |
| Lead II | 1140 | 1110 | 1104 |

TABLE 20
V₄, V₅, and V₆ signals

| | Positive electrode | Negative electrode | Ground electrode |
|---|---|---|---|
| V₄ signal | 1104 | 1110 | 1140 |
| V₅ signal | 1130 | 1110 | 1140 |
| V₆ signal | 1106 | 1110 | 1140 |

In preferred embodiments, electrodes 1104, 1130, and 1106 are respectively positioned on the precordial V₄, V₅, and V₆ positions. In such instances, the V₄, V₅ and V₆ signals are measured using any of the connections described in Tables 14 through 20 and the intermittent scheme described in Section 5.1, in conjunction with FIGS. 6 and 7.

In other embodiments, electrodes 1130 and 1106 are respectively positioned on the precordial V₄ and V₅ positions. In such instances, the V₄ and V₅ signals and lead II can be measured using the electrode configurations specified in Table 21.

TABLE 21
Electrodes 1130 and 1106 incorrectly positioned on the V₄ and V₅
positions, respectfully

| | Positive electrode | Negative electrode | Ground electrode |
|---|---|---|---|
| V₄ signal | 1130 | 1110 | 1104 |
| V₅ signal | 1106 | 1110 | 1104 |
| Lead II | 1140 | 1110 | 1104 |

A third possible outcome is that electrodes 1104 and 1130 are respectively positioned on the precordial V₅ and V₆ positions. In such instances, the V₅ and V₆ signals and lead II can be measured using the electrode configurations specified in Table 22.

TABLE 22
Electrodes 1104 and 1130 incorrectly positioned on the V₅ and V₆
positions, respectfully

| | Positive electrode | Negative electrode | Ground electrode |
|---|---|---|---|
| V₅ signal | 1104 | 1110 | 1106 |
| V₆ signal | 1130 | 1110 | 1106 |
| Lead II | 1140 | 1110 | 1104 |

### 5.5.2 Single lead ECG

FIG. 11B illustrates a two-electrode configuration in accordance with another embodiment of the present invention. In the electrode configuration, a first non-conductive pad 1190 and a second non-conductive pad 1108 are used. A first electrode 1192 is disposed on pad 1190. Electrodes 1192 is adapted for electrical connection with the skin of a subject 1120 in order to receive and transmit electrical impulses. In the embodiment illustrated in FIG. 11B, electrode 504 is placed any of the precordial positions V₁ through V₆. A second electrode 1110 is disposed on the second non-conductive pad 1108. Electrode 1110 is adapted for electrical connection with the skin in order to receive and transmit electrical impulses. Electrode 1110 is a right arm (RA) electrode that is for positioning at a position that is on or close to the right arm of subject 520. In some embodiments, electrode 1110 is placed on or near the S position depicted in FIG. 4.

Although not shown, an apparatus in accordance with the embodiment of the present invention illustrated in FIG. 5 includes an electrical connection that connects the electrodes disposed on pads 1108 and 1190 to an electrocardiological measuring apparatus. The two pad electrode system illustrated in FIG. 11 is highly advantageous because only two pads need to be positioned on the body. If pad 1190 is placed such that electrode 1192 overlies the V₄ precordial position (position 35, FIG. 3), then the V₄ signal is measured by the electrocardiological measuring apparatus. If, on the other hand, pad 1190 is placed such that electrode 1192 overlays the V₅ precordial position (position 45, FIG. 3), then the V₅ signal is measured by the electrocardiological measuring apparatus.

### 5.5.3 Additional Lead Combinations

In addition to the electrode configurations illustrated above, the present invention provides additional electrode configurations that are capable of measuring many different types of leads. Central to each of these electrode configurations is the minimization of the number of pads that are used to host the electrodes as well as the ability to automatically switch between the different leads supported by such electrode configurations so that the leads are measured without user intervention or assistance. Each of these additional electrode configurations is designed for one of the many different indications and applications that can be addressed by the present invention.

It is well known that distinct lead combinations are used to identify the specific location or site of ischemia, injury, or infarct. As used herein, an infarct is an area of necrosis in the heart resulting from obstruction of the local circulation by a thrombus or embolus. For example, heart anterior wall ischemia, injury, or infract is best detected using the V₃ and V₄ leads. Heart lateral wall ischemia, injury, or infarct is best measured using lead I, aVL, V₅, and V₆. Heart inferior wall ischemia, injury, or infarct is best measured using lead II, lead III, and aVF. Heart septal wall ischemia, injury, or infarct is best measured using V₁ and V₂. In addition, distinct lead combinations are used to measure occlusions. An occlusion is the blockage of a blood vessel. For example, leads V₁ through V₆ can be used to detect a left anterior descending (LAD) coronary artery occlusion. Lead I, aVL and possibly V₅ and V₆ can by used to detect a circumflex (Cx) coronary artery occlusion, and leads II, III, and aVF can be used to detect a right coronary artery (RCA) occlusion.

The reason that particular lead combinations are used to detect distinct ischemias, injuries, infarcts, and occlusions is that each lead measures a different portion of the heart. For example, leads I, aVL, V₅ and V₆ measure the lateral wall of the heart. Leads II, III, aVF, V₃, and V₄ measure the anterior wall of the heart. Leads V₁ and V₂ measure the septal wall of the heart. Further, leads V₃ and V₄ measure the anterior wall of the heart.

Configuration 1101 can be used to measure V₂ (or V₁) and V₅. Configuration 1101 places three electrodes on three pads. Electrode 1113 is positioned for RA, whereas electrode 1115 is placed at precordial position V₂ (or V₁) and electrode 1117 is placed at precordial position V₅. Electrodes 1113, 1115, and 1117 are connected to device 600 (FIG. 6). Then lead selection between the V₂ (or V₁) signal and the V₅ signal is automatically determined by ECG-lead switch 604. In particular ECG-lead switch 604 is used to collect both the V₂ (V₁) signal and the V₅ signal. This is accomplished by first collecting one of the two signals (V₂ or V₅) and then alternating the electrode assignment so that the other signal is collected. Advantageously, the switch between the V₂ (or V₁) and V₅ signals (or vice versa) is automatically accomplished by ECG-lead switch without intervention by the user.

Configuration 1103 can be used to measure the V₂ (or V₁) and the V₄ and the V₅ (or the V₅ and the V₆) leads. Configuration 1103 places four electrodes on three pads. Electrode 1113, on the first pad, is positioned for RA, whereas electrode 1115, on the second pad, is placed at precordial position V₂ (or precordial position V₁). Electrodes 1119 and 1121, on the third pad, are placed at one of two positions. They are either placed (i) at the V₄ and V₅ precordial positions respectively or (ii) at the V₅ and V₆ precordial positions respectively. Electrodes 1113, 1115, 1119 and 1121 are connected to device 600 (FIG. 6). Then lead selection between the V₂ signal (or the V₁ signal), the V₄ signal and the V₅ signal (or the V₅ and V₆ signals) is automatically determined by ECG-lead switch 604. In particular, ECG-lead switch 604 is used to collect each of these signals. This is accomplished by first collecting one of the signals and then alternating the electrode assignment so that another of the signals is collected. This process is repeated until each of the signals (leads) has been measured.

Configuration 1105 can be used to measure the V₂ (or V₁) and V₅ leads. Configuration 1105 places three electrodes on three pads. Electrode 1113, on the first pad, is positioned for RA, whereas electrode 1115, on the second pad, is placed at precordial position V₂ (or precordial position V₁). Electrode 1125, on the third pad, is placed at the V₅ precordial position. Electrodes 1113, 1115, and 1125 are connected to device 600 (FIG. 6). Then lead selection between the V₂ signal (or the V₁ signal depending on where electrode 1115 was placed) and the V₅ signal is automatically determined by ECG-lead switch 604. In other words, ECG-lead switch 604 is used to collect each of these signals (leads). This is accomplished by first collecting one of the signals and then alternating the electrode assignment so that the other signal is collected.

Configuration 1107 can be used to measure the V₂ (or V₁) lead, the V₄ and the V₅ (or the V₅ and the V₆) leads, and lead II. Configuration 1103 places five electrodes on four pads. Electrode 1113, on the first pad, is positioned for RA, whereas electrode 1115, on the second pad, is placed at precordial position V₂ (or precordial position V₁). Electrodes 1127 and 1129, on the third pad, are placed at one of two positions. They are either placed (i) at the V₄ and V₅ precordial positions respectively or (ii) at the V₅ and V₆ precordial positions respectively. Electrode 1131, on the fourth pad, is placed at electrode position LL. Electrodes 1113, 1115, 1127, 1129, and 1131 are connected to device 600 (FIG. 6). Then, lead selection between the V₂ signal (or the V₁ signal), the V₄ signal and the V₅ signal (or the V₅ and V₆ signals), and lead II are automatically determined by ECG-lead switch 604. Electrode configuration 1111 is identical to electrode configuration 1107 with the exception that electrodes 1127, 1129, and 1131 are combined onto a single pad to simplify the electrode configuration and make it easier for a user to properly arrange the electrodes. Electrode configuration 1109 is identical to electrode configuration 1111 with the exception that it includes only one of the precordial electrodes in the range V₄ through V₆ (i.e., one of V₄, V₅ and V₆).

### 5.6 Pre-Screening Management Services

Novel electrode configurations for measuring an ECG have been presented. The novel electrode configurations use a minimum number of leads and pads. Further, the novel electrode configurations are controlled by a digital signal processing system control 608 that can drive the same set of electrodes in alterative ways during respective discrete time intervals 702 in order to measure multiple signals (e.g., lead II, V₄, V₅). Thus, the electrode configurations and ECG measuring apparatus of the present invention do not need the assistance of specially trained medical professionals. This ECG method is highly suitable for a widespread pre-screening strategy to detect abnormal findings often associated with myocardial ischemia and/or silent myocardial infarction (SMI). For more information on risk factors see, for example, London and Kaplan, “Advances in electrocardiographic monitoring” in Kaplan, 3^{rd} edition, 1993, Cardiac Anesthesia, Philadelphia, WB Saunders, p. 300, Table 10-1, which is hereby incorporated by reference in its entirety.

One aspect of the present invention combines any of the novel electrode configurations described in Sections 5.1 through 5.5 with a system that includes ECG capture, communications, Internet, and server database technology integrated with automated data identification software to enable a disease management service for the general population as well as an interactive means to measure and manage individual heart health. The system effectively addresses the pre-screening of a large population concerning heart health that would be impractical and too costly to institute in the absence of the inventive systems.

### 5.6.1 Representative System

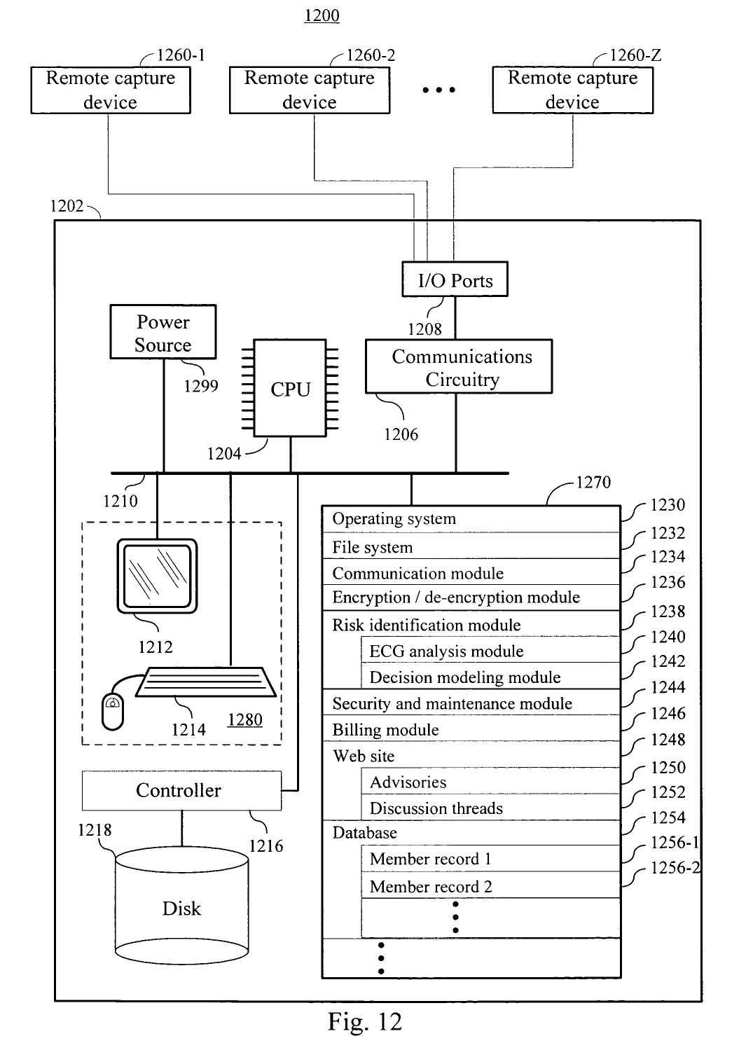

Referring to FIG. 12, a specific embodiment of a system 1200 in accordance with this aspect of the invention is illustrated. System 1200 preferably comprises a server 1202 that includes:
a central processing unit 1204;
a main non-volatile storage unit 1218, preferably including one or more hard disk drives, for storing software and data, the storage unit 1218 typically controlled by disk controller 1216;
a system memory 1270, preferably high speed random-access memory (RAM), for storing system control programs, data, and application programs, including programs and data loaded from non-volatile storage unit 1218; system memory 1270 can also include read-only memory (ROM);
an optional user interface 1280, including one or more input devices, such as a mouse, a keypad 1214, and/or a display 1212;
network interface circuitry 1206 for connecting to any wired or wireless communication network, the network interface circuitry 1206 optionally including a plurality of ports 1208;
one or more internal buses 1210 for interconnecting the aforementioned elements of the system; and
a power source 1299 for providing power to the above identified components.

Server 1202 is in communication with a plurality of devices 1260. Each device 1260 is a remote capture device in accordance with the present invention. As such, each remote capture device 1260 is capable of being configured to have any one of the electrode configurations described in Sections 5.1 through 5.5, above.

Operation of server 1202 is controlled primarily by operating system 1230, which is executed by central processing unit 1204. Operating system 1230 can be stored in system memory 1270. In addition to operating system 1230, a typical implementation of system memory 1270 includes:
file system 1232 for controlling access to the various files and data structures used by the present invention;
communication module 1234 for communicating with remote capture devices 1260;
an optional encryption/de-encryption module 1236 for decrypting data received from remote capture devices 1260 in embodiments where such devices encrypt transmitted data;
a risk identification module 1238 for processing data communicated from remote capture devices 1260 and stored in the database 1254;
an optional security and maintenance module 1244 for maintaining data security as well as for maintaining server 1202;
an optional billing module 1246 for billing organizations and/or subjects for services rendered by system 1200;
a web site 1248 for disseminating processed data relating to risk levels of subjects that have submitted medical data to server 1202 via remote capture devices 1202; and
a database 1254 for storing one or more records 1256 for each subject that submits medical data to server 1202 via remote capture devices 1202.

Database 1254 is any form of data storage system, including but not limited to, a flat file, a relational database (SQL), and an OLAP database (MDX and/or variants thereof). In some specific embodiments, database 1254 is a hierarchical OLAP cube. In some embodiments, there is only a single database 1254 while, in other embodiments, there are a plurality of databases 1254. In some embodiments of the present invention, system 1200 includes a plurality of servers 1202. The servers 1202 can be in one centralized location. However, more preferably, the servers 1202 are distributed over a large geographic area.

In one embodiment of the present invention, system 1200 is used to implement a method of identifying the risk that a subject has for coronary heart disease. Four different sources of information are used to enable this risk identification process (A) risk information from questionnaires, (B) physical information, (C) an ECG, and (D) blood tests.

A. Risk Information from questionnaires. Two types of information can be obtained from questionnaires (i) personal risk factor information and (ii) family history risk factor information. Personal factor information includes age, whether the subject smokes, exercise regimen and other measures of physical activity, height, weight, and diet. Some forms of personal risk factor information, including blood pressure, cholesterol level, and a test for diabetes require measurement but the user can still be queried for information on these subjects using the questionnaire. Of these risk factors, it is noted that smoking, physical activity, weight, diet, blood pressure, diabetic condition, and cholesterol level are modifiable conditions. Family history of risk factor information can also be derived from information provided by a subject using a guided questionnaire. Such information includes whether the subjects family has had coronary events, stroke, heart failure, peripheral vascular disease, diabetes, high blood pressure, or major vascular surgery.

In some embodiments, risk factor information is obtained using questionnaires presented by remote capture devices 1260 (FIG. 12). Further, such information can be collected at any participating care provider's office, government health clinics, employer sponsored health clinics, at any site that performs medical procedures, or in instances where personal physicals are needed (e.g., physicals required as a condition for new employment or to obtain life insurance).

In some embodiments, personal record information for the subject is obtained using, for example, such questionnaires. Representative personal record information includes, but is not limited to, a name, an address, a telephone number, an age, an ethnicity, or an e-mail address of the subject and risk factor information.

B. Physical Information. Physical information includes the measurement of vital signs such as blood pressure and heart rate. Such information can be collected at any participating care provider's office, government health clinics, employer sponsored health clinics, at any site that performs medical procedures, or in instances where personal physicals are needed (e.g., physicals required as a condition for new employment or to obtain life insurance).

C. Electrocardiograms. A central aspect of the present invention is the development of ECG systems that can be used to measure the ECG. Such systems have been described in previous sections. The ECG systems of the present invention are advantageous because they provide for the ability to collect multiple highly sensitive leads using a minimum pad configuration and an ECG-lead switch 604 coupled to an ECG signal digital signal processing system control that can automatically switch between measurement of such leads in order to improve sensitivity.

In one aspect of the invention, a electrocardiogram is obtained for a subject using remote capture device 1260. In some embodiments in accordance with this aspect of the invention, the remote capture device comprises a first non-conductive pad. A first electrode and a second electrode are disposed on the first non-conductive pad and they are adapted for electrical connection with the skin in order to receive and transmit electrical impulses. The first electrode represents any one of V₄, V₅, or V₆ and the second electrode is either (i) positioned on the subject below the first electrode in order to represent left leg (LL) or (ii) placed on a line on the subject defined by the V₄, V₅, and V₆ precordial positions in order to represent any one of V₄, V₅, or V₆ not represented by the first electrode. As used here, the term approximately below means that the second electrode is placed at some position below the precordial line. In some embodiments, the second electrode represents left leg and is positioned at least 12 cM from the heart. For a discussion on the placement of the left leg (LL) electrode, see Kaplan, Cardiac Anaesthesia, 3rd edition, 1993, pp. 326-327. In some embodiments, the first electrode represents V₄ or V₅ and is for positioning on V₄ or V₅ of the subject and the second electrode represents LL. The remote capture device further comprises a second non-conductive pad. A third electrode is disposed on the second non-conductive pad and is adapted for electrical connection with the skin in order to receive and transmit electrical impulses. The third electrode is a right arm (RA) electrode that is for positioning on or close to the right arm of the subject. Remote capture device 1260 further comprises an electrical connection that connects each electrode disposed on the first and the second non-conductive pad to an electrocardiological measuring apparatus such as device 600.

Because of the advantageous features of the ECG devices of the present invention, electrocardiograms can be obtained in a minimally assisted manner. Accordingly, the electrocardiogram data can be collected at any participating care provider's office, government health clinics, employer sponsored health clinics, at any site that performs medical procedures, or in instances where personal physicals are needed (e.g., physicals required as a condition for new employment or to obtain life insurance).

D. Blood tests. To complement other data used to make a coronary heart disease risk assessment, blood tests (e.g., cholesterol, high density lipoprotein/low density lipoprotein, etc) or other diagnostic tests (e.g., diabetes, etc.) are obtained. In addition, genetic markers that indicate that an individual is genetically predisposed to coronary heart disease can be detected using such tests. Exemplary types of markers include, but are not limited to, restriction fragment length polymorphisms “RFLPs”, random amplified polymorphic DNA “RAPDs”, amplified fragment length polymorphisms “AFLPs”, simple sequence repeats “SSRs”, single nucleotide polymorphisms “SNPs”, microsatellites, etc. Such data can be collected at government health clinics, employer sponsored health clinics, at any site that performs medical procedures, or in instances where personal physicals are needed (e.g., physicals required as a condition for new employment or to obtain life insurance).

Once the above information has been acquired and downloaded into database 1254, it is analyzed to identify risk factors associated with coronary heart disease. In some embodiments, identifying the data for asymptomatic CHD comprises performing an ECG analysis and then performing decision modeling based on the ECG reading. Typically, the ECG analysis determines ECG findings where the ECG findings are (i) an identification of no abnormal ECG findings or (ii) an identification of one or more abnormal ECG findings. The decision modeling module 1242 determines a pre-screening identification for the subject based on the risk factors stored and the ECG findings. In some instances, the stored information provides at least one risk factor such as at least one of an advanced age, a blood pressure, a cholesterol level, the results from a test for diabetes, lifestyle, a sex, or the ethnicity of the subject. In some embodiments, the method further comprises providing a report of the data using a web site. In some cases, the web site is secured with a user identification and a password associated with the subject.

The screening process identified above provides a two-fold screening strategy. First, subjects are screened for modifiable risk factors (e.g., high blood pressure, high cholesterol, diabetes, smoking, obesity, diet, etc.). Second, subjects are ECG screened to obtain baseline information, or to identify evidence of SMI or silent and/or inducible ischemia. Advantageously, the screening that is performed is the same method of screening that a cardiologist or primary care physician uses when assessing asymptomatic patients for coronary heart disease. What the present invention has accomplished is the same method of screening without the expense, inconvenience, and labor required for the cardiologist's or primary care physician's assessment. Therefore, the pre-screening process identified above can be used to identify at risk groups early in order to modify the risk factors and therefore reduce morbidity and mortality associated with coronary heart disease.

An overview of a system 1200 in accordance with one embodiment of the present invention has been presented. The system is highly advantageous because it allows for the widespread community-based pre-screening of subjects for risk factors associated with coronary artery disease. Further, a method for using system 1200 has been described. The following sections provide a more detailed description of individual components of exemplary system 1200.

### 5.6.2 Data Capture

Advantageously, the data capture process is decentralized by providing remote capture devices 1260 at point of care sites such as any medical or medically related offices, as well as other sites where sufficient instruction for data capture is available. Sites such as health spas, exercise centers, and even homes may be suited for such capture devices. It is anticipated that, in at least some instances, the subject will be experiencing physical, medically induced, or psychological stress when the remote ECG is administered. For example, in some instances, the patient will have the ECG administered after working out in an exercise center. In other instances, certain medical events and the drugs employed in treatment may cause stress to the heart due to a lack of oxygen supply (e.g., hypotension, low hemoglobin, hypoxia). In addition the physiological effects of the office visit can induce stress to the heart. Such stress conditions are normally not induced by treadmill exercises. These induced stresses can, in fact, improve the results of the applied ECG reading over a conventional resting ECG reading and is considered to be a benefit of the present invention.

System 1200 is beneficial for managed care organizations (MCOs) because the management of large population pre-screening data and the pre-screening costs are dramatically reduced. Specific CHD risks are identified early, resulting in lower intervention costs, and the collected data is in a form that is available to the physician without incurring additional collection costs. In addition, system 1200 provides the general population with interactive tools that allow them to constructively participate in managing their own heart care modifiable risk factors effectively, thereby assisting MCOs in minimizing subsequent health care costs.

In an embodiment of the present invention, subjects provide medical data to system 1200 in the form of a questionnaire that is electronically presented by remote capture devices 1260. Further, subjects use remote capture equipment to record an ECG. Remote capture devices 1260 are configured to provide any of the electrode configurations described in Sections 5.1 through 5.5, above. As such, proper ECG measurement using the remote capture devices does not require personnel trained in detailed ECG procedures. From the data collected from the subject, system 1200 identifies community members at risk for asymptomatic or symptomatic CHD.

In some embodiments, system 1200 identifies community members who
carry modifiable cardiac risk factors;
have a history of at-risk CHD;
show stress signs in the ECG collected using the remote capture devices of the present invention;
have symptomatic CHD but are unaware that symptoms are related to CHD and therefore would not normally come to a physician for a cardiac check-up;
have asymptomatic but significant CHD, also referred to as “silent myocardial infarction” (SMI);
show abnormal vital signs (high blood pressure, irregular heart beat, etc.); and/or
show actual CHD symptoms when under stress.

In some embodiments much of the pre-screening risk factor information is captured in the form of personal answers to specific life style questions. Yes or no, percentages, and frequencies are captured based on questions asked. The capture of such risk factor information involves an interactive relationship with a user very much like filling out a form in a physician's office. Such data capture is efficiently accomplished using remote capture devices 1260. Questions are presented to the user on a screen. In some embodiments, the screen is a part of the remote capture device 1260. In other embodiments, the remote capture device 1260 interfaces with a personal computer or personal digital assistant which is then used to present the questionnaire. In some embodiments of the present invention, remote capture device 1260 comprises a personal computer, a laptop, a personal digital assistant, a cell phone, or a related or equivalent electronic device. Regardless of the exact configuration of device 1260, the subject has the ability to either choose or input the answer, and move on to the next question until all the answers to the questions are captured.

In addition to the text-based questionnaire, a specific ECG test is also recorded. ECG sensors in any one of the configurations presented in Sections 5.1 through 5.5 are attached to the subject's body to record the ECG. In a preferred embodiment, remote capture device 1260 comprises a PDA style hand held unit that encompasses the ECG sensors, the question/answer screen, and the transmission capability to communicate the full screening information to server 1202. While ECG data collected using remote capture devices 1260 represents important screening information, other related medical tests are input when available such as the results of blood tests (e.g., cholesterol, high density lipoprotein/low density lipoprotein, etc) or other diagnostic tests (e.g., diabetes, etc.)

In addition to capturing the risk factor information, the remote capture devices 1260 receive identification information (e.g., name, address, age, phone, etc.) for the subject for tracking purposes. In some embodiments, such data is electronically available at the physician office setting, the subject's work setting, or at an insurance sponsored event. In such instances this information can be electronically communicated to the remote capture device 1260 and/or server 1202 so that such information does not have to be re-entered by the subject.

### 5.6.3 Communication of Data From Remote Devices to a Central Server

In typical embodiments, remote capture device 1260 communicates acquired data using an Internet connection between the device 1260 and server 1202. The Internet connection can be facilitated with a modem, DSL, or other form of connection between device 1260 and an Internet Service Provider (not shown), which in turn communicates data to server 1202 over the Internet. Furthermore, with the emergence of wireless application protocols (WAP), Internet-based data transmission to, and results from, server 1202 is far more portable, and therefore provides more convenience to subject. Applications of Bluetooth, 802.11b, and short messaging Service (SMS) can be invoked in order to facilitate an Internet-based connection between server 1202 and connections to an Internet portal without resorting to conventional wire hookup. See, for example, Networking Complete 2^{nd} edition, 2001, Sybex Inc. Alameda Calif., which is hereby incorporated by reference in its entirety.

In some embodiments data transferred from remote capture devices 1260 to server 1202 is encrypted. For instance, in some embodiments secret key cryptography, public key cryptography, or a hash algorithm is used to encrypt data transferred between devices 1260 and server 1202 over the Internet. When this is the case, server 1202 includes an encryption/de-encryption module in order to un-encrypt received medical data. For more information on representative encryption algorithms that can be used to transfer data in system 1200, see Kaufman et al., 1995, Network Security, Private Communication in a Public World, Prentice-Hall, Inc., Upper Saddle River, N.J., which is hereby incorporated by reference in its entirety.

### 5.6.4 Risk Identification Module

In one embodiment of the present invention, risk identification module 1238 comprises two modules, an ECG analysis module 1240, and a decision modeling module 1242.

ECG analysis module 1240 automatically analyzes the digital representation of captured ECG for each member. Module 1240 will report basic ECG results as “normal”, or report a set of indications that require additional analysis. This result has a direct impact on the identified risk for a subject in the population. In some embodiments, module 1240 is a software program such as RealTime 2.11 (Institute for Biosignal Engineering, Vienna, Austria).

Decision modeling module 1242 reads all relevant member information stored in the record 1256 associated with a subject and prioritizes the data in terms of screening parameters. This results in a pre-screening identification/unit time (score) for each subject. Then, the results are stored in the record 1256 for the subject.

### 5.6.5 Web Site

Web site 1248 is used to communicate and report the results of the data submitted by subjects after they have provided medical data using a remote capture device 1260. Subjects can also receive risk identification from system 1200 using cell phones and personal digital assistants (PDAs). In some embodiments, web site 1248 presents to a subject their personal screening status, participation in learning, advisories, and progress in taking charge of specific heart related health issues on a web page associated with the subject.

In some embodiments, access to a web page hosted by server 1202 requires a user identifier and a password that is periodically changed. In addition, under some circumstances, medical personnel are given access to information gathered for their patients by server 1202. In a preferred embodiment, access to patient information is only granted after the patient has approved such access.

Web site 1248 provides a number of services to subjects that have provided medical information, including an ECG, to server 1202. For example, the site can issue certain advisories 1250 based on patient-specific levels of risks to any of the risk factors associated with coronary artery disease. Furthermore, site 1248 can host discussion threads 1252 that are relevant to classes of tested subjects. For example, there can be a discussion thread 1252 for subjects that are at high risk for a particular risk factor, etc. Each discussion thread 1252 can serve as a forum for exchanging information to assist the subjects. In some embodiments, discussion threads 1252 are mediated by trained medical personnel to ensure the integrity of the information that is disseminated and shared in each discussion thread.

In some embodiments an identification of the risk factors associated with a subject and/or an analysis of the ECG data for the subject may lead to the need to refer the subject to a primary care physician for risk assessment and risk management. The primary care physician can assess the patient using the data acquired from the systems and methods of the present invention, perform additional blood tests, perform additional required assessment and/or other needed health screening. This information can optionally be stored in the member record 1256 (FIG. 12) associated with the subject so that proper advisories 1250 are sent to the subject. Additionally, the physician can manage the modifiable risk factors (e.g., high blood pressure, high cholesterol, diabetes, etc.) using specific methods of treatment, thereby lowering the risk for morbidity and mortality associated with coronary heart disease. In some instances, the primary care physician can refer the subject to a specialist, such as a cardiologist.

### 5.6.6 Database for Storing Medical Data

Database 1254 stores the medical data provided by subjects that use remote capture devices 1260 as well as all of the processed screening results. In some embodiments of the present invention, database 1254 has the following attributes: (i) Internet connected with specific WEB links, (ii) tight data privacy standards for access and retrieval, (iii) all information keyed from a unique member ID number, (iv) capable of receiving screening information either all at once or in segments from remote capture devices 1260. In some embodiments, database 1260 links all the information for a given subject together under the ID number for the subject. In some embodiments, each member record 1256 supports a time stamp data structure to enable evaluation over extended time periods. In some embodiments, the results of risk identification module 1238 are stored in database 1254.

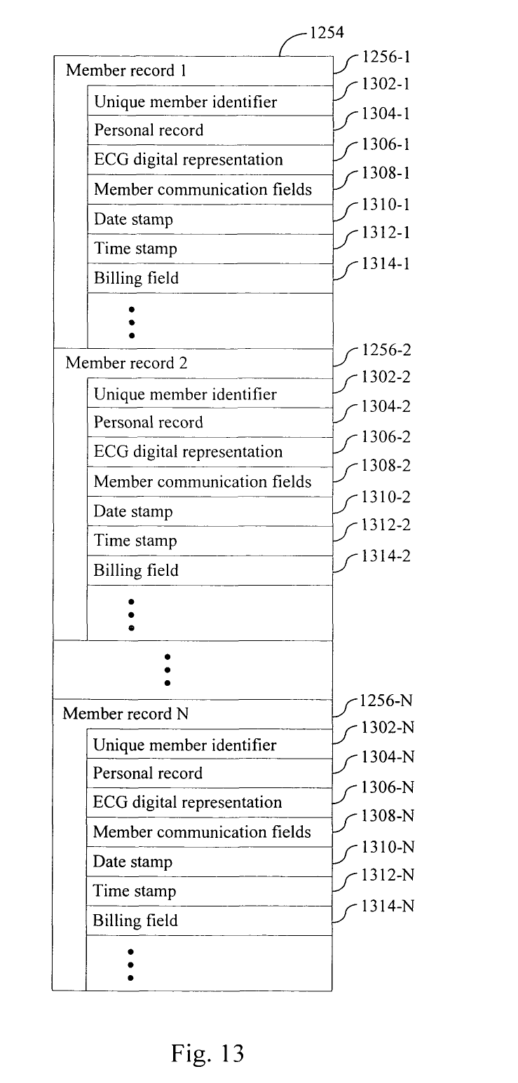

Referring to FIG. 13, in one embodiment of the present invention each member record 1256 is associated with a different subject. Further, each member record 1256 includes:
a unique member identifier 1302 for the subject that is associated with the record;
a member personal record 1304 that includes identification information such as the name, address, telephone number, age, e-mail address of the subject associated with the record, and the collected member risk factor information;
ECG data 1306 for storing a digital representation of the captured ECG data;
member communication fields 1308 for storing results from the risk identification module 1238 for the subject associated with the record, including status, alert, and other information for member reporting;
a date stamp 1310 and time stamp 1312 that is used to track when records are captured so that a history can be generated; and
one or more billing fields 1314 to help monitor billing for system 1200.

### 5.7 Sponsors

The systems and methods of the present invention provide a beneficial method for pre-screening a large population for risks associated with coronary heart disease. Although the systems and methods of the present invention can be used to administer an ECG to subjects at substantially reduced costs relative to known protocols, there is still a cost associated with the test. Accordingly, the present invention contemplates a number of sponsorship sources to defray costs associated with large scale screening of the members of a community. Patient care organizations are the primary care team that would provide additional screening services from at-risk population pre-screened by the systems and methods of the present invention. As such they would also provide direct patient communications via e-mail or Internet in order to have a dialog with the at-risk patients.

For any given community, there are, at a minimum, six sponsors who have a clear interest in the pre-screening of CHD in a general population using the systems and methods of the present invention: (i) individual community members willing to pay for the service, (ii) health maintenance organizations, (iii) insurance companies, (iv) corporate employers in the community, (v) local hospitals, and (vi) federal and local government agencies. Such sponsors will be discussed in the following subsections.

### 5.7.1 Individual Community Members

Through a strong marketing campaign, and through participating physician recommendations, patients are encouraged to use remote capture devices 1260 conveniently located in the physician's office. There are individuals who will recognize the value and convenience of the programs offered by the present invention and will pay to participate. For those individuals who choose to take an active role in determining and maintaining heart health, the system and methods of the present invention provide an efficient means to that end.

### 5.7.2 Health Maintenance Organizations (HMOs)

It is contemplated that HMOs will sponsor and support operation of systems such as system 1200 (FIG. 12) because it is a viable means to control costs. The current screening process relies on scheduling multiple office visits to perform a rest ECG test that often show negative results. The portable remote capture devices 1260 a sensitive, portable means for obtaining ECG data. Further such data can be obtained and subsequently managed with a marked decrease in overall expense relative to known systems. Further such data is obtained and subsequently managed in database 1254 where primary care physicians can access their patient's medical history data thereby realizing a marked decrease in overall managed care expense relative to known systems. In addition, database 1254 serves as an efficient means for recording, tracking, reminding and statistically reviewing the heart health of the member population. With this patient history captured and monitored, a new era of disease management is effectively enabled.

In some embodiments, only those HMO members who actually need treatment move into the sequential diagnostic and treatment sequences defined by the HMO. This cost savings, coupled with an enhanced public relations view gained by using the systems and methods of the present invention, provides a strong incentive to HMOs to make use of the present invention.

### 5.7.3 Insurance Companies

Most insurance companies contract with HMOs to help manage medical services and reduce costs. They have an obligation to their investors to determine the health risks of prospective policyholders, and often contract out to paramedic services to perform initial screening of applicants before issuing policies for health insurance or life insurance. It is expected that the insurance companies will find the pre-screening services provided by the systems and methods of the present invention are less costly, more efficient, and more accurate than current outsourced protocols.

### 5.7.4 Large Corporate Employers

It is expected that large corporations use the systems and methods of the present invention to pre-screen their employees as well as to provide a way of encouraging employees to participate in programs designed to improve heart health. Such endeavors will prevent lost time away from the job due to sickness as well as increase worker productivity.

### 5.7.5 Federal and Local Government

CHD is a malady that afflicts an aging population, and therefore advanced CHD treatment often becomes the responsibility of federal government sponsored Medicare. The systems and methods of the present invention provide the potential to reduce the instance and severity of CHD by early diagnosis. This will result in direct cost savings within the Medicare program.

State and local community governments can use the pre-screening interactive services of the present invention to improve heart health as well as broaden essential services to the community. While avoiding comprehensive government sponsored health programs, specific community outreach programs showing local benefits to the community in conjunction with other services can be initiated with local government sponsorship and support.

### 5.7.6 Local Hospitals

Local hospital systems concentrate on serving two groups who have a choice as to what hospital to select (i) the hospital consumer, and (ii) the physician.

The hospital consumer. The hospital consumer is concerned with the overall image the hospital presents to the community concerning quality health care. Hospitals where pervasive health problems such as cardiac care are emphasized are certainly factors in consumer selection. If the hospital takes active steps to extend their care influence to the outlying regions of the community where other forms of health care are sparse, it will improve the image of the hospital. Employing the pre-screening management services of the present invention is an effective way to serve outlying communities as well as serve the local community. Funding in the form of endowments are likely means to enable such outreach programs.

The physician. The local hospital system is constantly seeking and retaining physicians to be a part of their system. Physicians choose which hospital to work at based on many criteria. As such physicians share the same concerns considered by the hospital consumer described above. In addition, the physician is concerned about the hospital physical and medical environment, diagnostic equipment availability, and the quantity and quality of community outreach programs designed to identify and treat patients at the hospital. Employing the pre-screening management systems of the present invention in community outreach programs will help the hospital attract and retain qualified physicians.

### 5.8 Databases

Referring to FIG. 13, one aspect of the present invention provides a database 1254 having a member record 1256 for each subject in a plurality of subjects. Each member record includes: (i) a member identifier 1302 for the subject corresponding to the member record 1254, (ii) a personal record 1304 for the subject corresponding to the member record 1256 and all collected risk factor information, and (iii) ECG data 1306 for the subject corresponding to the member record 1256. ECG data 1306 is obtained by a remote capture device 1260 that has a first non-conductive pad. A first electrode and a second electrode are disposed on the first non-conductive pad and they are adapted for electrical connection with the skin of the subject in order to receive and transmit electrical impulses. The first electrode represents any one of V₄, V₅, or V₆ and the second electrode is either (i) positioned on the subject below the first electrode in order to represent left leg (LL) or (ii) placed on a line on the subject defined by the V₄, V₅, and V₆ precordial positions in order to represent any one of V₄, V₅, or V₆ not represented by the first electrode. In some embodiments, the first electrode represents V₄ or V₅ and is for positioning on V₄ or V₅ of a subject and the second electrode represents LL. Device 1260 includes a second non-conductive pad. A third electrode is disposed on the second non-conductive pad and is adapted for electrical connection with the skin of the subject in order to receive and transmit electrical impulses. The third electrode is a right arm (RA) electrode that is for positioning on or close to the right arm of the subject. An electrical connection connects each electrode disposed on the first and the second non-conductive pad to an electrocardiological measuring apparatus 600.

In some embodiments, a personal record 1304 in database 1254 includes the results of a blood test (e.g., cholesterol, high density lipoprotein/low density lipoprotein, etc) or other diagnostic tests (e.g., diabetes, etc.) associated with the member record 1256. In some embodiments, the personal record 1304 comprises a name, an address, a telephone number, an age, and/or an e-mail address for the subject corresponding to the member record 1256. In some embodiments, a personal record 1304 in the member record 1256 further comprises a pre-screening identification for the subject corresponding to the member record 1256. Typically, the pre-screening identification is based on results of the decision modeling module 1242. In some embodiments, a personal record 1304 in the member record 1256 further comprises all collected risk factor information for the subject associated with the member record 1256 such as an age of the subject, a blood pressure of the subject, a cholesterol level of the subject, the results from a test for diabetes for the subject, lifestyle of the subject, a sex of the subject, or the ethnicity of the subject.

### 5.9 References Cited

All references cited herein are incorporated herein by reference in their entirety and for all purposes to the same extent as if each individual publication or patent or patent application was specifically and individually indicated to be incorporated by reference in its entirety for all purposes.

### 5.10 Alternative Embodiments

The present invention can be implemented as a computer program product that comprises a computer program mechanism embedded in a computer readable storage medium. For instance, the computer program product could contain the program modules shown in FIG. 12. These program modules can be stored on a CD-ROM, magnetic disk storage product, or any other computer readable data or program storage product. The software modules in the computer program product can also be distributed electronically, via the Internet or otherwise, by transmission of a computer data signal (in which the software modules are embedded) on a carrier wave.

Those of skill in the art will appreciate that any of the module and databases depicted in memory 1270 of server 1202 including communication module 1234, encryption/de-encryption module 1236, risk identification module 1238, security and maintenance module 1244, billing module 1246, web site 1248, and/or database 1254 can, in fact, be stored on one or more remote computers.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. An apparatus for electrocardiogram measurement consisting of, in combination:
a first non-conductive pad;
a first electrode and a second electrode disposed on said first non-conductive pad and adapted for electrical connection with the skin of a subject in order to receive and transmit electrical impulses, wherein said first electrode represents any one of V₄, V₅, or V₆ and the second electrode is either (i) positioned on the subject below the first electrode in order to represent left leg (LL) or (ii) is placed on a line on the subject defined by the V₄, V₅, and V₆ precordial positions in order to represent any one of V₄, V₅, or V₆ not represented by the first electrode;
a second non-conductive pad;
a third electrode disposed on said second non-conductive pad and adapted for electrical connection with the skin in order to receive and transmit electrical impulses, wherein said third electrode is a right arm (RA) electrode that is for positioning at a position that is on or close to the right arm of said subject; and
an electrocardiological measuring apparatus that is in electrical communication with said first electrode, said second electrode, and said third electrode, wherein said electrocardiological measuring apparatus is capable of measuring both a first lead and a different second lead without user intervention.

2. The apparatus of claim 1 wherein said second electrode represents LL.

3. The apparatus of claim 2 wherein said electrocardiological measuring apparatus is configured to measure a V₄ or a V₅ lead from said third electrode (RA) and said first electrode, when said second electrode (LL) is ground.

4. The apparatus of claim 2 wherein said electrocardiological measuring apparatus is configured to measure lead II from said third electrode (RA) and said second electrode (LL) when said first electrode (V₄) is ground.

5. The method of claim 1 wherein said first lead is V₄ or V₅ and said second lead is lead II.

6. The apparatus of claim 1 wherein said second electrode represents V₅.

7. The apparatus of claim 6 wherein said electrocardiological measuring apparatus is configured to measure a V₄ lead from said third electrode (RA) and said first electrode (V₄) when said second electrode (V₅) is ground.

8. The apparatus of claim 6 wherein said electrocardiological measuring apparatus is configured to measure a V₅ lead from said third electrode (RA) and said second electrode (V₅) when said first electrode (V₄) is ground.

9. The apparatus of claim 1 wherein
said apparatus further consists of a fourth electrode disposed on said first non-conductive pad and adapted for electrical connection with the skin of the subject in order to receive and transmit electrical impulses,
said second electrode represents LL;
said fourth electrode represents V₅;
said second electrode is for positioning below said first electrode and said fourth electrode; and
said fourth electrode is in electrical communication with said electrocardiological measuring apparatus.

10. The apparatus of claim 9 wherein said electrocardiological measuring apparatus is configured to measure lead II from said third electrode (RA) and said second electrode (LL) when either said fourth electrode (V₅) or said first electrode (V₄) is ground.

11. The apparatus of claim 9 wherein said electrocardiological measuring apparatus is configured to measure a V₄ lead from said third electrode (RA) and said first electrode (V₄) when said fourth electrode (V₅) is ground.

12. The apparatus of claim 9 wherein said electrocardiological measuring apparatus is configured to measure a V₄ lead from said first electrode (V₄) and an aVL lead, where the aVL lead is taken from the third electrode (RA) and the second electrode (LL), when the fourth electrode (V₅) is ground.

13. The method of claim 9 wherein said first lead is V₄ or V₅ and said second lead is aVL.

14. The apparatus of claim 9 wherein said electrocardiological measuring apparatus is configured to measure a V₄ lead from said third electrode (RA) and said second electrode (LL) when said fourth electrode (V₅) is ground.

15. The apparatus of claim 9 wherein said electrocardiological measuring apparatus is configured to measure a V₅ lead from said third electrode (RA) and said fourth electrode (V₅) when said first electrode (V₄) is ground.

16. The apparatus of claim 9 wherein said electrocardiological measuring apparatus is configured to measure a V₅ lead from said fourth electrode (V₅) and an aVL signal, where the aVL signal is taken from the third electrode (RA) and the second electrode (LL), when the first electrode (V₄) is ground.

17. The apparatus of claim 1 wherein said apparatus further consists of a fourth electrode disposed on said first non-conductive pad and adapted for electrical connection with the skin in order to receive and transmit electrical impulses, wherein said second electrode represents V₅ and said fourth electrode represents V₆ and wherein said fourth electrode is in electrical communication with said electrocardiological measuring apparatus.

18. The apparatus of claim 17 wherein said electrocardiological measuring apparatus is configured to measure a V₄ lead from said third electrode (RA) and said first electrode (V₄) when said fourth electrode (V₆) or said second electrode (V₅) is ground.

19. The apparatus of claim 17 wherein said electrocardiological measuring apparatus is configured to measure a V₅ lead from said third electrode (RA) and said second electrode (V₅) when said fourth electrode (V₆) or said first electrode (V₄) is ground.

20. The apparatus of claim 17 wherein said electrocardiological measuring apparatus is configured to measure a V₆ lead from said third electrode (RA) and said fourth electrode (V₆) when said second electrode (V₅) or said first electrode (V₄) is ground.

21. The method of claim 17 wherein said first lead and said different second lead are each selected from the group consisting of V₄, V₅, and V₆.

22. The apparatus of claim 1 wherein
said apparatus further consists of a fourth electrode and a fifth electrode disposed on said first non-conductive pad and adapted for electrical connection with the skin in order to receive and transmit electrical impulses;
said second electrode represents V₅;
said fourth electrode represents V₆;
said fifth electrode represents LL and is for positioning below said first, second, and fourth electrodes; and
said fourth and said fifth electrodes are in electrical communication with said electrocardiological measuring apparatus

23. The apparatus of claim 22 wherein said electrocardiological measuring apparatus is configured to measure lead II from said third electrode (RA) and said fifth electrode (LL) when said first electrode (V₄), said second electrode (V₅), or said fourth electrode (V₆) is ground.

24. The apparatus of claim 22 wherein said electrocardiological measuring apparatus is configured to measure a V₄ lead from said third electrode (RA) and said first electrode (V₄) when said fourth electrode (V₆) or said second electrode (V₅) is ground.

25. The apparatus of claim 22 wherein said electrocardiological measuring apparatus is configured to measure a V₄ lead from first electrode (V₄) and an aVL lead, where the aVL lead is taken from the third electrode (RA) and the fifth electrode (LL), when said second electrode (V₅) or said fourth electrode (V₆) is ground.

26. The apparatus of claim 22 wherein said electrocardiological measuring apparatus is configured to measure a V₅ lead from said third electrode (RA) and said second electrode (V₅) when said fourth electrode (V₆) is ground.

27. The apparatus of claim 22 wherein said electrocardiological measuring apparatus is configured to measure a V₅ lead from said second electrode (V₅) and an aVL lead, where the aVL lead is taken from the third electrode (RA) and the fifth electrode (LL), when said fourth electrode (V₆) or said first electrode (V₄) is ground.

28. The apparatus of claim 22 wherein said electrocardiological measuring apparatus is configured to measure a V₆ lead from said third electrode (RA) and said fourth electrode (V₆) when said second electrode (V₅) is ground.

29. The apparatus of claim 22 wherein said electrocardiological measuring apparatus is configured to measure a V₆ lead from said fourth electrode (V₆) and an aVL lead, where the aVL lead is taken from the third electrode (RA) and the fifth electrode (LL), when said second electrode (V₅) or said first electrode (V₄) is ground.

30. The apparatus of claim 1 wherein said first electrode represents V₄ or V₅ and is for positioning on V₄ or V₅ of said subject and wherein said second electrode represents LL.

31. The connector device of claim 1, wherein said first non-conductive pad and said second non-conductive pad are selected from non-conductive natural or synthetic material.

32. A method of identifying asymptomatic coronary heart disease (CHD) for a subject, the method comprising:
(A) obtaining an electrocardiogram measurement from said subject using a remote device consisting of, in combination:
a first non-conductive pad;
a first electrode and a second electrode disposed on said first non-conductive pad and adapted for electrical connection with the skin in order to receive and transmit electrical impulses, wherein said first electrode represents any one of V₄, V₅, or V₆ and the second electrode is either (i) positioned on the subject below the first electrode in order to represent left leg (LL) or (ii) is placed on a line on the subject defined by the V₄, V₅, and V₆ precordial positions in order to represent any one of V₄, V₅, or V₆ not represented by the first electrode; and
a second non-conductive pad;
a third electrode disposed on said second non-conductive pad and adapted for electrical connection with the skin in order to receive and transmit electrical impulses, wherein said third electrode is a right arm (RA) electrode that is for positioning on or close to the right arm of said subject; and
an electrocardiological measuring apparatus that is in electrical communication with said first electrode, said second electrode, and said third electrode, wherein said electrocardiological measuring apparatus is capable of measuring both a first lead and a different second lead without user intervention; and
(B) analyzing said electrocardiogram measurement.

33. The method of claim 32, the method further comprising collecting potential risk factor information from said subject through manual or electronic interrogation means.

34. The method of claim 33 wherein the potential risk factor information is collected in the form of a questionnaire.

35. The method of claim 32, the method further comprising obtaining any one or more of results of a diagnostic test for said subject.

36. The method of claim 32, the method further comprising obtaining personal record information for said subject.

37. The method of claim 36 wherein said personal record information comprises any one or more of a name, an address, a telephone number, an age, an ethnicity, and an e-mail address.

38. The method of claim 32 wherein said analyzing said electrocardiogram measurement comprises:
performing an ECG analysis; and
performing decision modeling based on said ECG analysis.

39. The method of claim 38 wherein said performing ECG analysis determines ECG findings, wherein said ECG findings are an identification of no abnormal ECG finding or an identification of one or more abnormal ECG findings.

40. The method of claim 39 wherein said decision modeling determines a pre-screening identification for said subject based on a function of said ECG findings.

41. The method of claim 40 wherein said pre-screening identification is further determined by considering a risk factor.

42. The method of claim 41 wherein said risk factor is at least one of an advanced age of the subject, the blood pressure of the subject, the cholesterol level of the subject, the results from a test for diabetes of the subject, the sex of the subject, whether the subject smokes, an assessment of the physical activity of the subject, the weight of the subject, the diet of the subject and the ethnicity of the subject.

43. The method of claim 32, the method further comprising providing a report of said electrocardiogram measurement using a web site.

44. The method of claim 43 wherein said web site is secured with a user identification and a password associated with said subject.

45. The method of claim 32 wherein said first electrode represents V₄ or V₅ and is for positioning on V₄ or V₅ of said subject and wherein said second electrode represents LL.

46. The method of claim 39 wherein said ECG findings are any one or more of silent myocardial infarction (SMI), silent ischemia, and inducible ischemia.

47. The method of claim 32 wherein said first lead is V₄ or V₅ and said second lead is lead II.

48. A computer system for identifying asymptomatic coronary heart disease (CHD) for a subject, the computer system comprising:
a central processing unit;
a memory, coupled to the central processing unit, the memory storing:
(A) instructions for receiving data from a remote capture device; wherein said data is generated for a subject by a device consisting of, in combination:
a first non-conductive pad;
a first electrode and a second electrode disposed on said first non-conductive pad and adapted for electrical connection with the skin of the subject in order to receive and transmit electrical impulses, wherein said first electrode represents any one of V₄, V₅, or V₆ and the second electrode is either (i) positioned on the subject below the first electrode in order to represent left leg (LL) or (ii) is placed on a line on the subject defined by the V₄, V₅, and V₆ precordial positions in order to represent any one of V₄, V₅, or V₆ not represented by the first electrode; and
a second non-conductive pad;
a third electrode disposed on said second non-conductive pad and adapted for electrical connection with the skin in order to receive and transmit electrical impulses, wherein said third electrode is a right arm (RA) electrode that is for positioning on or close to the right arm of said subject; and
an electrocardiological measuring apparatus that is in electrical communication with said first electrode, said second electrode, and said third electrode, wherein said electrocardiological measuring apparatus is capable of measuring both a first lead and a different second lead without user intervention; and
(B) instructions for analyzing said data.

49. The computer system of claim 48, wherein said instructions for receiving further comprise instructions for receiving risk factor information and said instructions for analyzing further comprise instructions for analyzing said risk factor information.

50. The computer system of claim 48 wherein said data comprises ECG data.

51. The computer system of claim 50 wherein said ECG data is digitized.

52. The computer system claim of 48 wherein said data further includes risk factor information in the form of one or more of advanced age, cigarette smoking, hypertension, obesity, diabetic condition, high cholesterol, diet, family history, ethnicity, and sex for said subject.

53. The computer system of claim 48 wherein said data further comprises any one or more of results of a diagnostic test for said subject.

54. The computer system of claim 48 wherein said data further comprises personal record information for said subject.

55. The computer system of claim 54 wherein said personal record information comprises any one or more of a name, an address, a telephone number, an age, or an e-mail address for said subject.

56. The computer system claim 54 wherein said personal record information comprises any one or more of risk factor information in the form of advanced age, cigarette smoking, hypertension, obesity, diabetic condition, high cholesterol, diet, family history, ethnicity, and sex for said subject.

57. The computer system of claim 48 wherein said data is encrypted.

58. The computer system of claim 48 wherein said instructions for analyzing comprise:
instructions for performing ECG analysis; and
instructions for performing decision modeling.

59. The computer system of claim 58 wherein said instructions for performing ECG analysis determines ECG findings, wherein said ECG findings are an identification of no abnormal ECG findings or an identification of one or more abnormal ECG findings for said subject.

60. The computer system of claim 59 wherein said instructions for performing decision modeling determine a pre-screening identification for said subject based on a function of said ECG findings.

61. The computer system of claim 60 wherein said pre-screening identification is further determined by considering a risk factor for said subject.

62. The computer system of claim 61 wherein said risk factor is at least one of an advanced age of the subject, a blood pressure of the subject, a cholesterol level of the subject, the results from a test for diabetes for said subject, the sex of the subject, whether the subject smokes, an assessment of the physical activity of the subject, the weight of the subject, the diet of the subject, and the ethnicity of the subject.

63. The computer system of claim 48 wherein said memory further comprises a web site module, the web site module comprising instructions for providing a web site that includes a report of said data.

64. The computer system of claim 63 wherein said memory further comprises instructions for securing said web site with a user identification and a password associated with said subject.

65. The computer system of claim 48 the memory further comprising a database having a member record for each subject in a plurality of subjects, each member record comprising:
a member identifier for the subject corresponding to said member record;
a personal record for the subject corresponding to said member record wherein said personal record was obtained by said instructions for receiving data; and
ECG data for the subject corresponding to said member record wherein said ECG data was obtained by said instructions for receiving data;

66. The computer system of claim 65 wherein a personal record in said database further comprises risk factor information for the subject associated with the personal record in the form of a blood pressure of the subject, a cholesterol level of the subject, the results from a test for diabetes for the subject, the sex of the subject, whether the subject smokes, an assessment of the physical activity of the subject, the weight of the subject, the diet of the subject, and the ethnicity of the subject.

67. The computer system of claim 66 wherein said personal record further comprises any one or more of a name, an address, a telephone number, an age, or an e-mail address for the subject corresponding to said member record.

68. The computer system of claim 66 wherein said personal record further comprises a pre-screening identification for said subject corresponding to said personal record.

69. The computer system of claim 66 wherein a personal record in said database further comprises a risk factor for the subject associated with said personal record, wherein said risk factor comprises at least one of an advanced age of the subject, the blood pressure of the subject, the cholesterol level of the subject, the results from a test for diabetes, the sex of the subject, whether the subject smokes, an assessment of the physical activity of the subject, the weight of the subject, the diet of the subject, and the ethnicity of the subject.

70. The computer system of claim 48 wherein said first electrode represents V₄ or V₅ and is for positioning on V₄ or V₅ of said subject and wherein said second electrode represents LL.

71. The computer system of claim 59 wherein said ECG findings is any one or more of abnormal findings, or evidence of silent myocardial infarction (SMI), silent ischemia, or inducible ischemia.

72. A database having a member record for each subject in a plurality of subjects, each member record comprising
a member identifier for the subject corresponding to said member record;
a personal record for the subject corresponding to said member record;
ECG data for the subject corresponding to said member record wherein said ECG data is obtained by a remote capture device consisting of, in combination:
a first non-conductive pad;
a first electrode and a second electrode disposed on said first non-conductive pad and adapted for electrical connection with the skin of said subject in order to receive and transmit electrical impulses, wherein said first electrode represents any one of V₄, V₅, or V₆ and the second electrode is either (i) positioned on the subject below the first electrode in order to represent left leg (LL) or (ii) is placed on a line on the subject defined by the V₄, V₅, and V₆ precordial positions in order to represent any one of V₄, V₅, or V₆ not represented by the first electrode;
a second non-conductive pad;
a third electrode disposed on said second non-conductive pad and adapted for electrical connection with the skin in order to receive and transmit electrical impulses, wherein said third electrode is a right arm (RA) electrode that is for positioning on or close to the right arm of the subject; and
an electrocardiological measuring apparatus that is in electrical communication with said first electrode, said second electrode, and said third electrode, wherein said electrocardiological measuring apparatus is capable of measuring both a first lead and a different second lead without user intervention.

73. The database of claim 72 wherein a personal record in said database comprises the results of a diagnostic tests for the subject associated with said member record.

74. The database of claim 72 wherein a personal record in said database comprises any one or more of a name, an address, a telephone number, an age, or an e-mail address for the subject corresponding to said member record.

75. The database of claim 72 wherein a personal record in said database comprises a pre-screening identification for said subject corresponding to said member record, wherein a value of said pre-screening identification is based on an analysis of said ECG data.

76. The database of claim 72 wherein a personal record in said database further comprises risk factor information for the subject associated with said member record, wherein said risk factor information comprises at least one of an advanced age of said subject, a blood pressure of said subject, a cholesterol level of said subject, the results from a test for diabetes for said subject, a sex of said subject, or the ethnicity of said subject.

77. The database of claim 72 wherein said first electrode represents V₄ or V₅ and is for positioning on V₄ or V₅ of a subject and wherein said second electrode represents LL.

78. A method of identifying asymptomatic coronary heart disease (CHD) for a subject, the method comprising:
(A) collecting risk factor information from said subject;
(B) obtaining an electrocardiogram measurement from said subject using a remote device consisting of, in combination:
a first non-conductive pad;
a first electrode and a second electrode disposed on said first non-conductive pad and adapted for electrical connection with the skin in order to receive and transmit electrical impulses, wherein said first electrode represents any one of V₄, V₅, or V₆ and the second electrode is either (i) positioned on the subject below the first electrode in order to represent left leg (LL) or (ii) is placed on a line on the subject defined by the V₄, V₅, and V₆ precordial positions in order to represent any one of V₄, V₅, or V₆ not represented by the first electrode; and
a second non-conductive pad;
a third electrode disposed on said second non-conductive pad and adapted for electrical connection with the skin in order to receive and transmit electrical impulses, wherein said third electrode is a right arm (RA) electrode that is for positioning on or close to the right arm of said subject; and
an electrocardiological measuring apparatus that is in electrical communication with said first electrode, said second electrode, and said third electrode, wherein said electrocardiological measuring apparatus is capable of measuring both a first lead and a different second lead without user intervention; and
(C) analyzing said collected risk factor information and said electrocardiogram measurement.

79. The method of claim 78 wherein said risk factor information is collected from said subject in the form of a questionnaire.

80. The method of claim 78 wherein said analyzing comprises:
performing an ECG analysis of said electrocardiogram measurement; and
performing decision modeling based on said ECG analysis.

81. The method of claim 80 wherein said decision modeling determines a pre-screening identification for said subject based on a function of said ECG analysis.

82. The method of claim 81 wherein said pre-screening identification is further determined by considering said collected risk factor information.

83. The method of claim 82 wherein said risk factor information comprises at least one of an advanced age of the subject, the blood pressure of the subject, the cholesterol level of the subject, the results from a test for diabetes of the subject, the sex of the subject, whether the subject smokes, an assessment of the physical activity of the subject, the weight of the subject, the diet of the subject and the ethnicity of the subject.

84. A computer system for identifying a risk factor for a subject, the computer system comprising:
a central processing unit;
a memory, coupled to the central processing unit, the memory storing:
(A) instructions for receiving data from a remote capture device; wherein said data comprises risk factor information and ECG data for said subject;
(B) instructions for analyzing said data for said risk factor; and
(C) instructions for storage of said data and a result of said instructions for analyzing; wherein said ECG data is measured by a device consisting of, in combination:
a first non-conductive pad;
a first electrode and a second electrode disposed on said first non-conductive pad and adapted for electrical connection with the skin of the subject in order to receive and transmit electrical impulses, wherein said first electrode represents any one of V₄, V₅, or V₆ and the second electrode is either (i) positioned on the subject below the first electrode in order to represent left leg (LL) or (ii) is placed on a line on the subject defined by the V₄, V₅, and V₆ precordial positions in order to represent any one of V₄, V₅, or V₆ not represented by the first electrode; and
a second non-conductive pad;
a third electrode disposed on said second non-conductive pad and adapted for electrical connection with the skin in order to receive and transmit electrical impulses, wherein said third electrode is a right arm (RA) electrode that is for positioning on or close to the right arm of said subject; and
an electrocardiological measuring apparatus that is in electrical communication with said first electrode, said second electrode, and said third electrode, wherein said electrocardiological measuring apparatus is capable of measuring both a first lead and a different second lead without user intervention.
